(19) United States
(12) Patent Application Publication
(10) Pub. No.: US 2026/0018240 A1
(43) Pub. Date: Jan. 15, 2026

(54) EQUIVARIANT DIFFUSION MODEL FOR GENERATIVE PROTEIN DESIGN

(71) Applicants: Genentech, Inc., South San Francisco, CA (US); Hoffmann-La Roche Inc., Little Falls, NJ (US)

(72) Inventors: Richard A. BONNEAU, New York, NY (US); Kyunghyun CHO, New York, NY (US); Vladimir GLIGORIJEVIC, New Brunswick, NJ (US); Andreas LOUKAS, Basel (CH); Jan Marcin LUDWICZAK, Basel (CH); Karolis MARTINKUS, Basel (CH)

(21) Appl. No.: 19/278,505

(22) Filed: Jul. 23, 2025

Related U.S. Application Data
(63) Continuation of application No. PCT/US2024/012903, filed on Jan. 25, 2024.
(60) Provisional application No. 63/481,776, filed on Jan. 26, 2023.
(60) Provisional application No. 63/501,107, filed on May 9, 2023.
(60) Provisional application No. 63/589,207, filed on Oct. 10, 2023.

(51) Int. Cl. G16B 15/20; G16B 30/00; G16B 40/20

(52) U.S. Cl. CPC G16B 15/20; G16B 30/00; G16B 40/20

(57) ABSTRACT

A method may include applying a protein design computation model to generate an output sequence and an output three-dimensional structure of an output protein molecule by jointly denoising an input sequence and an input three-dimensional structure of an input protein molecule. The joint denoising may include modifying the input sequence by inserting, deleting, or changing the type of one or more constituent amino acid residues while performing corresponding updates to the positions of the atoms in each amino acid residue. The protein design computation model may operate on a fixed size representation of the input protein molecule. Prior and/or subsequent to the joint denoising, the protein design computation model may modify the input three-dimensional structure to conform to bond constraints. Moreover, an informative prior data distribution may be incorporated by training the protein design computation model on training samples with noise sampled from the informative prior data distribution.

### CROSS REFERENCE TO RELATED APPLICATIONS

This application claims priority to U.S. Provisional Application No. 63/481,776, entitled “EQUIVARIANT DIFFUSION MODEL FOR GENERATIVE PROTEIN DESIGN” and filed on Jan. 26, 2023, U.S. Provisional Application No. 63/501,107, entitled “EQUIVARIANT DIFFUSION MODEL FOR GENERATIVE PROTEIN DESIGN” and filed on May 9, 2023, and U.S. Provisional Application No. 63/589,207, entitled “EQUIVARIANT DIFFUSION MODEL FOR GENERATIVE PROTEIN DESIGN” and filed on Oct. 10, 2023, the disclosures of which are incorporated herein by reference in their entireties.

### TECHNICAL FIELD

The subject matter described herein relates generally to protein design and more specifically to a diffusion model for generating protein sequences and corresponding three-dimensional structures in atomic resolution.

### INTRODUCTION

Proteins are genetically encoded macromolecules whose diversity in size and chemical composition give rise to a gamut of functionalities. For example, by regulating biological systems, proteins facilitate many essential cellular functions including, for example, enzymatic reactions, molecular transport, regulation and execution of a number of biological pathways, cell growth, proliferation, nutrient uptake, morphology, motility, intercellular communication, and/or the like. A protein molecule may include one or more polypeptides, each of which including a sequence of amino acid residues linked together by peptide bonds (e.g., covalent peptide bonds). The amino acid residues that are encoded directly by the genetic code are called standard or canonical amino acid residues. Of the twenty canonical amino acid residues, each is formed by the same backbone atoms (e.g., an amino group (NH2), an alpha carbon (C_{α}), and a carboxylic group (COOH)) coupled with a different combination of side chain atoms (or R groups).

The primary structure of a protein molecule refers to the sequence of amino acid residues in each of the polypeptide chains forming the protein molecule. The backbone atoms in adjacent amino acid residues that participate in the peptide bonds (e.g., covalent peptide bonds) therebetween form a repeating sequence of atoms known as the polypeptide backbone (or backbone) of the protein molecule. The local folded structures (e.g., a helixes, β pleated sheet, and/or the like) that form within an individual polypeptide chain due to interactions between the backbone atoms (e.g., amino hydrogen atoms, carboxyl oxygen atoms, and/or the like) are referred to as the secondary structure of the protein molecule. Further interactions (e.g., non-covalent bonds such as hydrogen bonding, ionic bonding, dipole-dipole interactions, and van der Waals forces) between the side chains (or R-groups) of the amino acid residues in the protein molecule may cause individual polypeptide chains to fold, thus forming the tertiary structure of the protein molecule. The tertiary structure of the protein molecule is also known as the conformation or the three-dimensional structure of the protein molecule. In protein molecules having multiple polypeptide chains, the protein molecule may also exhibit a quaternary structure, which is formed when the polypeptide chains are packed and held together by hydrogen bonds and van der Waals forces (e.g., between nonpolar side chains).

The functions of a protein molecule may be contingent upon the sequence of amino acids in the polypeptide chains forming the protein molecule as well as the three-dimensional structure adopted by the polypeptide chains. For example, the primary structure of the protein molecule may determine the three-dimensional structure assumed by the protein molecule through the folding of the constituent polypeptide chains. In some cases, the binding affinity of the protein molecule towards a target molecule, such as a viral or tumor antigen, may depend on whether the polypeptide chains in the protein molecule are able to assume a three-dimensional structure that complements that of the target molecule and is sufficiently stable to support a binding interaction between the two molecules. As such, one notable objective of computational protein design is to construct one or more protein sequences (e.g., antibodies and/or the like) that exhibit certain desirable properties, including the ability to adopt a particular three-dimensional structure.

### SUMMARY

Systems, methods, and articles of manufacture, including computer program products, are provided for generative protein design in which a diffusion model is applied for generating protein sequence and structure. In one aspect, there is provided a system for generative protein design that includes at least one processor and at least one memory. The at least one memory may include program code that provides operations when executed by the at least one processor. The operations may include: receiving an input protein molecule including an input sequence and an input three-dimensional structure, the input sequence including a plurality of residues, and the input three-dimensional structure including a position of a plurality of atoms forming each residue included in the input sequence; generating a representation of the input protein molecule including the input sequence and the input three-dimensional structure; and applying a protein design computation model to generate, based at least on the representation of the input protein molecule, an output protein molecule, the protein design computation model generating the protein molecule by at least performing a joint denoising of the input sequence and the input three-dimensional structure to generate an output sequence and an output three-dimensional structure of the output protein molecule.

In another aspect, there is provided a method for generative protein design. The method may include: receiving an input protein molecule including an input sequence and an input three-dimensional structure, the input sequence including a plurality of residues, and the input three-dimensional structure including a position of a plurality of atoms forming each residue included in the input sequence; generating a representation of the input protein molecule including the input sequence and the input three-dimensional structure; and applying a protein design computation model to generate, based at least on the representation of the input protein molecule, an output protein molecule, the protein design computation model generating the protein molecule by at least performing a joint denoising of the input sequence and the input three-dimensional structure to generate an output sequence and an output three-dimensional structure of the output protein molecule.

In another aspect, there is provided a computer program product including a non-transitory computer readable medium storing instructions. The instructions may cause operations may executed by at least one data processor. The operations may include: receiving an input protein molecule including an input sequence and an input three-dimensional structure, the input sequence including a plurality of residues, and the input three-dimensional structure including a position of a plurality of atoms forming each residue included in the input sequence; generating a representation of the input protein molecule including the input sequence and the input three-dimensional structure; and applying a protein design computation model to generate, based at least on the representation of the input protein molecule, an output protein molecule, the protein design computation model generating the protein molecule by at least performing a joint denoising of the input sequence and the input three-dimensional structure to generate an output sequence and an output three-dimensional structure of the output protein molecule.

In some variations, one or more features disclosed herein including the following features can optionally be included in any feasible combination.

In some variations, the representation of the input protein molecule is a matrix having a plurality of rows, and wherein each row in the matrix corresponds to a spot in the input sequence.

In some variations, the matrix includes one column populated by an encoding of a type of residue occupying each spot in the input sequence and a column populated by one or more coordinates for each possible atom forming a residue.

In some variations, the representation of the input protein molecule is a fixed size representation having a same quantity of rows and/or a same quantity of columns for different length input sequences.

In some variations, the representation of the input protein molecule is generated by at least assigning, to each residue included in the input sequence, an integer position corresponding to a structural role of the residue.

In some variations, the representation of the input protein molecule includes a gap character for each integer position where the input sequence fails to include a residue having a corresponding structural role.

In some variations, the representation of the input protein molecule is generated by at least determining, based at least on a first position of each atom in one or more adjacent residues, a second position of each nonexistent atom associated with the gap character.

In some variations, the protein design computation model jointly denoises the input sequence and the input three-dimensional structure over a plurality of successive denoising steps.

In some variations, the protein design computation model jointly denoises the input sequence and the input three-dimensional structure by at least removing a first portion of noise at a first step before removing a second portion of noise at a second step.

In some variations, the protein design computation model generates the output sequence to be invariant to special Euclidean group SE(3) transformations and the output three-dimensional structure to be equivariant to special Euclidean group SE(3) transformations.

In some variations, the protein design computation model jointly denoises a plurality of frames in which each frame corresponds to the input three-dimensional structure being oriented in one of two possible directions along each of two principal axes of rotation about a centroid of the input three-dimensional structure. The protein design computation model generates the output three-dimensional structure by at least averaging a result of jointly denoising the plurality of frames.

In some variations, the plurality of frames includes a first frame in which the input three-dimensional structure is oriented in one direction along a first principal axis of rotation, a second frame in which the input three-dimensional structure is oriented in an opposite direction along the first principal axis of rotation, a third frame in which the input three-dimensional structure is oriented in one direction along a second principal axis of rotation, and a fourth frame in which the input three-dimensional structure is oriented in the opposite direction along the second principal axis of rotation.

In some variations, the protein design computation model determines, based at least on a first direction of the input three-dimensional structure along the first principal axis of rotation and a second direction of the input three-dimensional structure along the second principal axis of rotation, a third direction of the input three-dimensional structure along a third principal axis of rotation.

In some variations, the protein design computation model includes a plurality of blocks, wherein each block of the protein design computation model includes a first multilayer perceptron (MLP) and a second multilayer perceptron (MLP), and wherein the plurality of blocks are applied consecutively to the representation of the input protein molecule.

In some variations, the protein design computation model further includes a projection layer, and wherein the projection layer modifies, based on one or more bond constraints, the input three-dimensional structure prior and/or subsequent to the joint denoising.

In some variations, the one or more bond constraints are imposed based on a reference residue backbone comprising a plurality of rigid atoms with fixed bond lengths and fixed bond angles.

In some variations, the projection layer modifies the input three-dimensional structure by at least determining one or more transformations that minimize a distance between the plurality of rigid atoms in the reference residue backbone and one or more backbone atoms in the input three-dimensional structure, and applying the one or more transformations to align the input three-dimensional structure to the reference residue backbone.

In some variations, the protein design computation model jointly denoises generic side chains comprising pseudo atoms having a same degrees of freedom as atoms in side chains of actual residues. The one or more bond constraints are imposed by at least replacing, subsequent to the joint denoising, the generic side chains with a side chain template of each type of residue forming the input three-dimensional structure.

In some variations, the projection layer modifies the input three-dimensional structure subsequent to the joint denoising by at least applying a dihedral angle between one or more pseudo atoms in the generic side chain template to a corresponding side chain template.

In some variations, an informative prior data distribution corresponding to a generative task of the protein design computation model may be determined. The informative prior data distribution may be incorporated by at least generating, based at least on the informative prior data distribution, one or more training samples for the protein design computation model.

In some variations, the generative task of the protein design computation model includes generating one or more protein molecules from a specific protein family.

In some variations, the informative prior data distribution includes a positional residue frequency specifying a likelihood of different types of residues occupying each spot in a protein sequence.

In some variations, the informative prior data distribution includes a conditional atom dependencies specifying a relative position of atoms in adjacent residues.

In some variations, the one or more training samples are generated to by at least adding, to one or more training protein molecules, noise sampled from the informative prior data distribution.

In some variations, the noise includes one or more modifications to at least one of a residue type and atomic positions.

In some variations, the one or more training samples are generated by a forward diffusion process that includes an incremental addition of the noise sampled from the informative prior data distribution.

In some variations, the input protein molecule exhibits one or more desirable properties, and wherein the protein design computation model generates the output protein molecule to also exhibit the one or more desirable properties.

In some variations, the one or more desirable properties include expression, binding affinity towards a target molecule, specificity, stability, non-immunogenicity, human-ness, and/or lack of self-association.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the computational design of protein molecules including protein-based therapeutics such as antibodies, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a protein design system, in accordance with some example embodiments;

FIG. 2A depicts a flowchart illustrating an example of a process for protein design, in accordance with some example embodiments;

FIG. 2B depicts a flowchart illustrating an example of a process for protein design, in accordance with some example embodiments;

FIG. 3 depicts a flowchart illustrating another example of a process for protein design, in accordance with some example embodiments;

FIG. 4 depicts a flowchart illustrating an example of a process for training a protein design computation model, in accordance with some example embodiments;

FIG. 5A depicts a schematic diagram illustrating an example architecture of a protein design computation model, in accordance with some example embodiments;

FIG. 5B depicts a schematic diagram illustrating an example representation of the sequence and three-dimensional structure of a protein molecule, in accordance with some example embodiments;

FIG. 6 depicts a schematic diagram illustrating an example of the three-dimensional structure of a protein molecule being oriented in three-dimensional space, in accordance with some example embodiments;

FIG. 7 depicts schematic diagrams of the full-atom representation of the amino acid residue lysine, tyrosine, and valine, and examples of the corresponding generic representation, in accordance with some example embodiments;

FIG. 8 depicts a schematic diagram illustrating the positional amino acid residue frequency observed in immunoglobulin protein molecules (or antibodies), in accordance with some example embodiments.

FIG. 9B depicts an example of an adjacency matrix specifying conditional atom dependencies, in accordance with some example embodiments

FIG. 10A depicts graphs illustrating the in vitro validation of in silico designed protein molecules in terms of expression, binding affinity, and binding rate, in accordance with some example embodiments;

FIG. 10B depicts the structure of the target molecule human epidermal growth factor receptor 2 (HER2) and the three-dimensional structures of the in silico generated binders, in accordance with some example embodiments;

FIG. 11 depicts a block diagram illustrating an example of a computing system, in accordance with some example embodiments.

When practical, similar reference numbers denote similar structures, features, or elements.

### DETAILED DESCRIPTION

Computational protein design aims to generate protein sequences that exhibit a variety of desirable properties. In the context of large molecule drug discovery (LMDD), the properties of a protein sequence may determine its viability as a protein-based therapeutic such as antibodies, enzymes, growth factors, hormones, interferons, interleukins, thrombolytics, and/or the like. Accordingly, as a protein-based therapeutic, a protein sequence may be computationally engineered for affinity and targetability, in vivo stability, pharmacokinetics, cell permeability, and non-immunogenicity. Computational protein design is a challenging and resource intensive task at least because protein molecules exhibit countless variations in sequence and conformation (or three-dimensional structure) but only a small proportion of these variants will have any therapeutic value. At the outset, of the 20^{L} possible protein sequences formed by an L-quantity of amino acid residues selected from the twenty canonical amino acid residues, few will have the drug-like properties (e.g., affinity, specificity, biological activity, and developability) required for a protein-based therapeutic. Moreover, many critical properties of a protein molecule, such as its binding affinity towards a target molecule, are also contingent on the three-dimensional structure formed by the folding of the underlying sequence of amino acid residue. Thus, in addition to navigating numerous variations in possible protein sequences, computational protein design is further complicated by the need to accurately predict, from amongst a myriad of possibilities, the three-dimensional structure of individual protein sequences. For instance, even if each of the L-quantity of amino acid residue in a protein sequence is confined to an N-quantity of discrete geometric states (e.g., rotamers), predicting the three-dimensional structure of the protein sequence may still entail exploring up to N^{L} possibilities.

Due to the sheer number of possible protein sequences and conformations, a naïve design approach that relies on a brute force examination of every possible sequence and conformational variation is computationally intractable for protein sequences of meaningful length (e.g., 110 amino acid residues in the variable domain of an antibody). However, indiscriminate efforts to reduce computational burden, for example, by assessing a few random selections of possible protein sequences, may overlook protein sequences with better properties than those in the existing repertoire. Meanwhile, given the link between protein sequence and three-dimensional structure, segregating sequence design and structural analysis may also yield suboptimal outcomes. For example, in cases where a language-based model is applied to generate protein sequences that then undergo separate structural predictions, those protein sequences are less likely to exhibit desirable properties, such as binding affinity and stability, at least because the language-based model lacks an awareness of the structural characteristics that contribute to these properties. Accordingly, various embodiments of the present disclosure provide for a protein design engine that implements a co-design approach to jointly determine the sequence (e.g., the type or identity of the amino acid residue occupying each position) and three-dimensional structure (e.g., the positions of the constituent atoms) of a protein molecule. By integrating protein sequence design and structural analysis to explore sequence and conformational variations in a strategic and resource efficient manner, the protein design engine may generate protein sequences that are capable of adopting suitable three-dimensional structures, such as a three-dimensional structure that complements the three-dimensional structure of a target molecule.

In some example embodiments, the protein design engine may apply a protein design computation model to co-design the sequence and three-dimensional structure of a protein molecule. Instead of a complex architecture based on transformers or graph neural networks, which can be memory inefficient and difficult to train, various embodiments of the protein design computation model described herein may be realized using one or more artificial neural networks (ANNs) such as a multilayer perceptron (MLP) and/or the like. In some cases, the protein design computation model may generate, based on an input protein molecule including an input sequence and a corresponding input three-dimensional structure, an output protein molecule including an output sequence and a corresponding output three-dimensional structure For example, in some cases, the output sequence and the output three-dimensional structure of the output protein molecule may be generated by at least denoising, through diffusion over successive step, the input sequence and the input three-dimensional structure of the input protein molecule. In some cases, the denoising may be incremental in that the protein design computation model removes a first portion of noise from the input protein molecule at a first step before removing a second portion of noise at a second step. Furthermore, in some cases, the protein design computation model may generate the output protein molecule by jointly denoising the input sequence and the input three-dimensional structure. For instance, in some cases, the joint denoising may include the protein design computation model modifying the input sequence by inserting, deleting, or changing the type of one or more constituent amino acid residues while performing corresponding updates to the positions (e.g., Cartesian coordinates (x, y, z)) of the atoms in each amino acid residue. In some cases, the protein design computation model may perform an equivariant diffusion of the atomic positions included in the input three-dimensional structure. That is, while the protein design computation model may generate the output three-dimensional structure to exhibit the same Euclidean transformations (e.g., translations, rotations, reflections, and/or the like) as the input three-dimensional structure, the updates that are made to the atomic positions in the input three-dimensional structure when generating the output three-dimensional structure may be unaffected by these Euclidean transformations. In some cases, the protein design computation model may also perform an invariant diffusion of the input sequence, meaning that the protein design computation model may disregard the Euclidean transformations present in the input three-dimensional structure when inserting, deleting, and/or changing the type of one or more amino acid residues in the input sequence to form the output sequence.

In some example embodiments, the protein design computation model may denoise the input protein molecule by operating on a tabular representation (or matrix representation) of the input protein molecule that represents the input sequence as well as the input three-dimensional structure of the input protein molecule. For example, in some cases, each row in the tabular representation of the input protein molecule may correspond to an amino acid residue in the input protein molecule. The type (or identity) of the amino acid residue (e.g., a one-hot encoding of the residue type) may populate one column of the tabular representation of the input protein molecule while the remaining columns may be populated by the positions of the atoms forming each amino acid residue. In some cases, the remaining columns in the tabular representation of the input protein molecule may include the positions of the different possible types of atoms (e.g., a carbon, β carbon, oxygen, nitrogen, and/or the like) that can form an amino acid residue. In cases where the positions of each atoms is defined by multidimensional coordinates (e.g., three-dimensional Cartesian coordinates (x, y, z)), the tabular representation of the input protein molecule may include a separate channel for each dimension. Where a particular type of atom is absent from an amino acid residue, the position of the atom in the corresponding entry (or entries) in the tabular representation of the input protein molecule may be determined (e.g., interpolated, extrapolated, and/or the like) based on the positions of the atoms that are present in the amino acid residue. For instance, in some cases, the position (e.g., three-dimensional Cartesian coordinates (x, y, z)) of a nonexistent atom in an amino acid residue may be determined (e.g., interpolated as the midway point, extrapolated by the addition a certain distance, and/or the like) based on the positions (e.g., three-dimensional Cartesian coordinates (x, y, z)) of one or more existent atoms (e.g., one or more nearest atoms, one or more atoms within a threshold distance, and/or the like). As described in more details below, where an actual amino acid residue is absent from a fixed length representation of the input sequence, the tabular representation of the input protein molecule may include a gap character indicative of a nonexistent amino acid residue. In some cases, the positions of the atoms in the nonexistent amino acid residue included in the tabular representation may be determined (e.g., interpolated, extrapolated, and/or the like) based on the positions of the atoms in one or more existent amino acid residues (e.g., one or more nearest amino acid residues in space, one or more adjacent amino acid residues in sequence, one or more amino acid residues within a threshold distance, and/or the like).

In some example embodiments, the protein design computation model may achieve equivariance to Euclidean transformations, particularly the rotations included in the special Euclidean group SE(3), through frame averaging. That is, the output three-dimensional structure generated by the protein design computation model may be rendered equivariant to various Euclidean transformations by at least averaging the outputs of the incremental denoising performed at each step by the protein design computation model across a subset of possible Euclidean transformations called frames. For example, in some cases, the protein design computation model may ingest four frames, each of which corresponding to the input three-dimensional structure being oriented in one of two possible directions along two out of the three principal axes of rotation about the centroid of the input three-dimensional structure. One of two possible directions in the third axis of rotation may be considered in order to respect the fixed chirality of the input three-dimensional structure, which can have non-superimposable (or non-identical) mirror images. For instance, in some cases, equivariance to rotations may be achieved by averaging, at each step, the output of the protein design computation model denoising each of the four aforementioned frames. That is, in some cases, the output of the protein design computation model at each step may be an average of the protein design computation model denoising a first frame in which the input three-dimensional structure is oriented in one direction along a first principal axis of rotation, a second frame in which the input three-dimensional structure is oriented in the opposite direction along the first principal axis of rotation, a third frame in which the input three-dimensional structure is oriented in one direction along a second principal axis of rotation, and a fourth frame in which the input three-dimensional structure is oriented in the opposite direction along the second principal axis of rotation. In this context, the term “average” and the operation of “averaging” may include the mean, median, and/or mode of the outputs across the selected frames. Accordingly, the output three-dimensional structure generated by the protein design computation model incrementally denoising the input three-dimensional structure over multiple successive steps may remain equivariant to the rotations included in the special Euclidean group SE(3) but not to the reflections that are also included in the Euclidean group E(3).

In some example embodiments, the protein design computation model may ingest a fixed-size representation (e.g., fixed-size tabular representation) of the input protein molecule in order to accommodate any length changes that occur as a part of the generative diffusion process. In this context, a length change may occur during the generative diffusion process when the denoising performed by the protein design computation model includes the insertion and/or deletion of one or more amino acid residues in the input sequence. While length changes are typically reflected in variations in the length of the input sequence, doing so may require the protein design computation model to be implemented with more computationally complex machine learning architectures, such as transformers and graph neural networks (GNNs), that impose greater memory burdens. Contrastingly, the fixed-size representation (e.g., fixed-size tabular representation) of the input protein molecule may allow the protein design computation model to be implemented using less computationally complex machine learning architectures, thus reducing the memory burden associated with the generative diffusion process. For example, in some cases, the protein design computation model may be implemented using one or more feed forward artificial neural networks (ANNs), such as one or more multilayer perceptrons (MLPs), which have linear memory complexity dependent on the quantity of rows and columns in fixed-size representation (e.g., fixed-size tabular representation) of the input protein molecule. Alternatively, the protein design computation model may also be implemented using other machine learning architecture including, for example, a convolutional neural network (e.g., a 1-dimensional convolutional neural network), a transformer, and/or the like.

In some example embodiments, the input sequence may be rendered in a fixed length representation by applying a structural role based numbering scheme in which each amino acid residue in the input sequence is assigned an integer corresponding to a particular structural role in the fixed length representation. For example, the input sequence may undergo structural alignment such that each amino acid present in the input sequence is aligned with a structural role and assigned a corresponding integer (e.g., selected from a range of integers such as [1, 149]). In instances where the input sequence corresponds to an immunoglobulin protein molecule (or antibody) or a portion thereof (e.g., the variable domain), these structural roles may correspond to different locations in various regions of the immunoglobulin protein such as a particular complementarity determining region (CDR) loop, a framework region between a pair of complementarity determining region (CDR) loops, and/or the like. A gap may be present in the fixed length representation of the input protein molecule anywhere in the input sequence where an amino acid residue having the corresponding structural role is absent. Accordingly, in some cases, a gap character may occupy one or more spots in the fixed length representation of the protein molecule where the input sequence lacks an amino acid residue having the corresponding structural role. In some cases, the insertion of an amino acid residue at a particular spot in the input sequence may be realized by replacing the gap character at the spot with the amino acid residue. Meanwhile, the deletion of an amino acid residue at a particular spot in the input sequence may be realized by replacing the amino acid residue at the spot with a gap character.

In some example embodiments, the generative diffusion process performed by the protein design computation model may be tailored to a specific task by incorporating an informative prior data distribution associated with the task. That is, instead of the input protein molecule having a uniform distribution or isotropic Gaussian distribution of noise in the type of amino acid residue and the atomic positions across every amino acid residue, the noise that is present in the input protein molecule may be task specific. For example, in instances where the protein design computation model is applied to generate proteins molecules from a particular protein family (e.g., immunoglobulin proteins (or antibodies) and/or the like), the generative diffusion process performed by the protein design computation model may incorporate an informative prior data distribution that reflects certain characteristics (e.g., residue-type frequency, conditional atom dependencies, and/or the like) present in the sequence and/or three-dimensional structure of protein molecules in the family. In some cases, the informative prior data distribution may have a small distance (e.g., Wasserstein distance and/or the like) relative to the posterior data distribution, which is the true data distribution being approximated by the protein design computation model in order to sample the output protein molecule therefrom. As such, incorporating the informative prior data distribution may reduce the complexity of denoising performed by the protein design computation model. For instance, in some cases, the informative prior data distribution may include noise in the type of amino acid residue corresponding to a frequency at which different residue types are observed in known protein molecules or a certain subset thereof (e.g., immunoglobulin protein molecules (or antibodies) and/or the like). Alternatively and/or additionally, the informative prior data distribution may include noise in atomic positions corresponding to a correlation between the positions of atoms forming neighboring amino acid residues (e.g., amino acid residues that are no more than n spots apart). Incorporating an informative prior data distribution instead of a noninformative data distribution (e.g., Gaussian (or normal) distribution, uniform distribution, and/or the like), which reduces the complexity of the denoising performed by the protein design computation model, may simplify learning, expedite generation, and improve data efficiency.

In some example embodiments, the protein design computation model may update the positions of the atoms in the input three-dimensional structure freely in three-dimensional space during the generative diffusion process while still imposing one or more bond constraints in order to ensure that the atoms in the output three-dimensional structure of the output protein molecule generated by the protein design computation model abide by the strong structural constraints found in nature. It should be appreciated that moving the atoms freely during the generative diffusion process may be more computationally efficient than the protein design computation model operating in an angle space in which the positions of the atoms in individual amino acid residues are represented in terms of the rotation and translation of rigid frames formed by adjacent atoms. In some cases, the protein design computation model may include a projection layer that modifies, based at least on the one or more bond constraints, the positions of the atoms in the input three-dimensional structure prior and/or subsequent to the updating of the atomic positions therein. For example, in some cases, the one or more bond constraints may be imposed based on a reference residue backbone in which the backbone atoms (e.g., carbon, α-carbon, nitrogen, and β-carbon) are positioned with idealized bond lengths and idealized bond angles therebetween. The projection layer may modify the positions of the backbone atoms in the input three-dimensional structure by at least transforming, for example, through one or more roto-translations, the positions of the backbone atoms to align with the atoms in the reference residue backbone. Alternatively and/or additionally, the one or more bond constraints may be imposed by based on one or more side chain templates, which are specific to each type of amino acid residue present in the output protein molecule. Because the type of each amino acid residue is unknown during the generative diffusion process, the protein design computation model may operate on a generic representation of each side chain until the type of every amino acid residue in the output protein molecule is determined. For instance, to represent a maximum four bonds capable of rotating around dihedral angles, the generic representation of a side chain may include four pseudo atoms (e.g., pseudo carbon atoms) having the same degrees of rotational freedom as those in the side chain of an actual amino acid residue. Upon generating the output protein molecule in which the type of each amino acid residue present in the output three-dimensional structure is identified, the projection layer may replace the generic side chain representation of each amino acid residue in the output sequence with the side chain template of the corresponding type of amino acid residue. Moreover, in some cases, the dihedral angles in each side chain template in the output three-dimensional structure may be replaced with the dihedral angles between the pseudo atoms in the generic side chain representation that had occupied the same spot in the output sequence.

In some example embodiments, the protein design computation model may perform the aforementioned generative diffusion process to generate the output protein molecule for a variety of applications. For example, the input protein molecule may be a member of a particular protein family (e.g., immunoglobulins (or antibodies) and/or the like) or a subset exhibiting certain desirable properties (e.g., expression, binding affinity, specificity, and/or the like), in which case the protein design computation model may generate the output protein molecule to be an additional member of the same protein family. Alternatively and/or additionally, the input protein molecule may exhibit certain desirable properties, such as binding affinity or specificity to a target molecule. In some cases, the protein design computation model may generate, based at least on the input protein molecule, the output protein molecule to exhibit same desirable properties, further increase the magnitude or the desirable properties, and/or exhibit additional desirable properties (e.g., expression, humanness, non-immunogenicity, and/or the like).

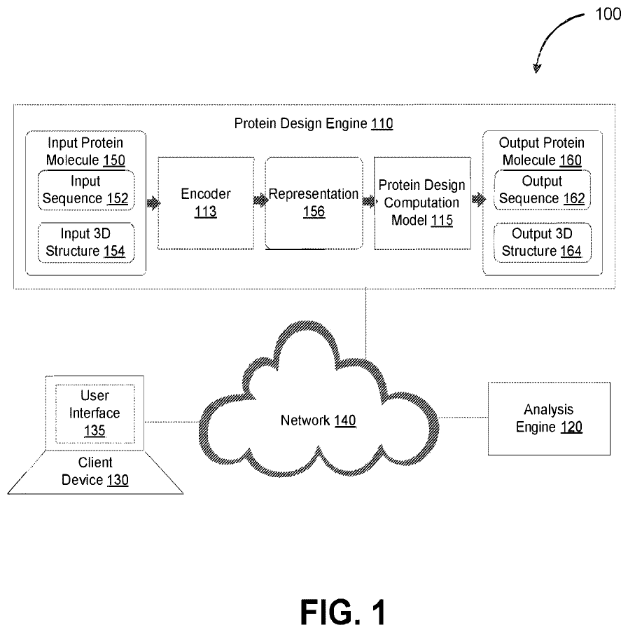

FIG. 1 depicts a system diagram illustrating an example of a protein design system 100, in accordance with some example embodiments. Referring to FIG. 1, the protein design system 100 may include a protein design engine 110, an analysis engine 120, and a client device 130. As shown in FIG. 1, the protein design engine 110, the analysis engine 120, and the client device 130 may be communicatively coupled via a network 140. The client device 130 may be a processor-based device including, for example, a workstation, a desktop computer, a laptop computer, a smartphone, a tablet computer, a wearable apparatus, and/or the like. The network 140 may be a wired network and/or a wireless network including, for example, a local area network (LAN), a virtual local area network (VLAN), a wide area network (WAN), a public land mobile network (PLMN), the Internet, and/or the like.

In some example embodiments, the protein design engine 110 may include an encoder 113 and a protein design computation model 115. For example, as shown in FIG. 1, the encoder 113 may generate, based at least on an input protein molecule 150 including an input sequence 152 and an input three-dimensional structure 154, a representation 156 of the input protein molecule 150. As described in more details below, in some cases, the encoder 113 may apply a structural role based numbering scheme such that the representation 156 is a fixed size representation of the input protein molecule 150, meaning that the size of the representation 156 remains the same regardless of the number of amino acid residues in the input sequence 152. For instance, in some cases, the representation 156 of the input protein molecule 150 may include one or more gap characters where the input sequence 152 lacks an amino acid residue having a certain structural role. Moreover, as described in more details below, the representation 156 of the input protein molecule 150 may be a tabular representation (or a matrix representation) in which each row corresponds to a spot in the input sequence 152 and is populated by the type (or identity) of the amino acid residue occupying the spot as well as the positions of the constituent atoms. The protein design computation model 115 may ingest the representation 156 of the input protein molecule 150 and generate an output protein molecule 160 that includes, for example, an output sequence 162 and an output three-dimensional structure 164 of the output protein molecule 160.

As will be described in more detail below, in some cases, the protein design computation model 115 may be a diffusion model trained to perform a joint denoising of the input sequence 152 and the input three-dimensional structure 154 of the input protein molecule 150 in order to generate the output sequence 162 and the output three-dimensional structure 164 of the output protein molecule 160. In some cases, the protein design computation model 115 may generate the output sequence 162 by at least performing an invariant diffusion on the input sequence 152, meaning that the protein design computation model 115 may disregard the Euclidean transformations (e.g., rotations, translations, and/or the like) present in the input three-dimensional structure 154 when inserting, deleting, and/or changing the type of amino acid residues present in the input sequence 152 such that the types of amino acid residues identified by the protein design computation model 115 as present in the output sequence 162 are unaffected by the Euclidean transformations in the input three-dimensional structure 154. Furthermore, in some cases, the protein design computation model 115 may perform an equivariant diffusion on the input three-dimensional structure 154 such that the output three-dimensional structure 164 exhibits the same Euclidean transformations (e.g., translations, rotations, reflections, and/or the like) as the input three-dimensional structure 154.

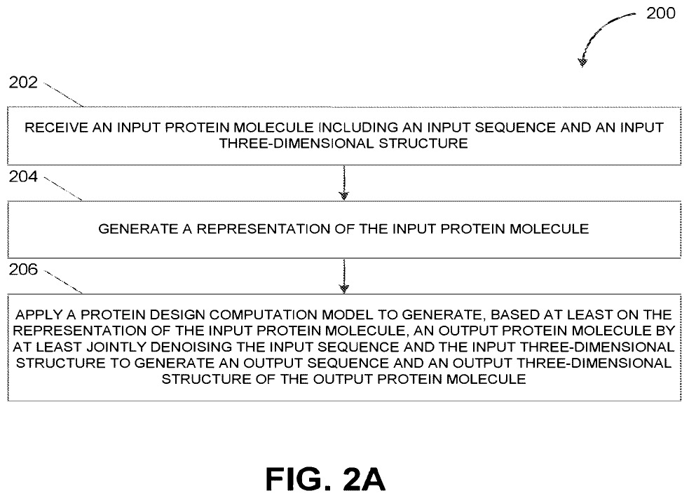

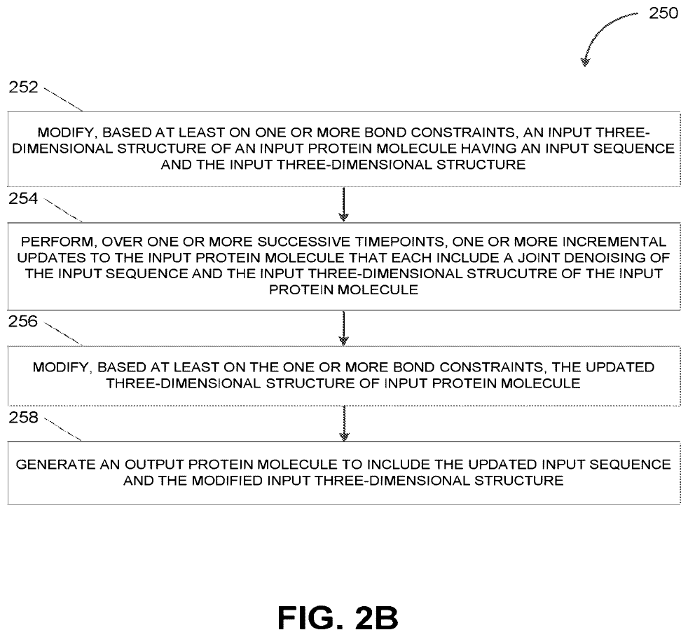

FIG. 2A depicts a flowchart illustrating an example of a process 200 for protein design, in accordance with some example embodiments. Referring to FIGS. 1 and 2A, the process 200 may be performed by the protein design engine 110 to generate, for example, the output protein molecule 160 based on the input protein molecule 150. In some cases, the protein design engine 110 may apply the protein design computation model 115, which may generate the output protein sequence 160 by jointly diffusing the input sequence 152 and the input three-dimensional structure 154. As described in more details below, the protein design computation model 115 may operate on the representation 156 of the input protein molecule 150, which may be a fixed-size representation (e.g., a fixed-size tabular representation) of the input protein molecule 150 capable of accommodating the length changes that occur during the generative diffusion process with greater computational efficiency. The protein design computation model 115 may also be implemented with one or more artificial neural networks (ANNs) (e.g., multilayer perceptrons (MLPs) and/or the like), which are easier train and impose less memory burdens than more computationally complex architectures such as graph neural networks (GNNs) and transformers.

At 202, the protein design engine 110 may receive an input protein molecule including an input sequence and an input three-dimensional structure. For example, in some cases, the protein design computation model 110 may receive the input protein molecule 150. The input sequence 152 of the input protein molecule 150 may include multiple amino acid residues, with each spot in the input sequence 152 being occupied, for example, by one of the twenty canonical amino acid residues. The input three-dimensional structure 154 of the input protein molecule 150 may include the position of one or more of the atoms forming each amino acid residue included in the input sequence 152. For instance, in some cases, the input three-dimensional structure 154 of the input protein molecule 150 may include the coordinates (e.g., Cartesian coordinates (x, y, z)) specifying the position of at least some of the atoms (e.g., heavy atoms) forming the individual amino acid residues in the input sequence 152.

At 204, the protein design engine 110 may generate a representation of the input protein molecule including the input sequence and the input three-dimensional structure. In some example embodiments, the encoder 113 of the protein design engine 110 may generate the representation 156 of the input protein molecule 150 such that the protein design computation model 115 operates on the representation 156 of the input protein molecule 150 when jointly diffusing the input sequence 152 and the input three-dimensional structure 154 to generate the output sequence 162 and the output three-dimensional structure 164 of the output protein molecule 160. In some cases, the representation 156 may be a fixed sized representation of the input protein molecule 150, meaning that the size of the representation 156 remains the same regardless of the size of the input protein molecule 150. For instance, in some cases, the size of the input protein molecule 150 may be defined by the length of the input sequence 152, which corresponds to the quantity of amino acid residues included in the input sequence 152. Fixing the size of the representation 156 of the input protein sequence 150 may allow the protein design computation model 115 to accommodate length changes during the generative diffusion process such that the length of (or the quantity of amino acid residues in) the output sequence 162 generated by the protein design computation model 115 is flexible. Contrastingly, conventional generative models require the quantity of amino acid residues in the output sequence 162 to be specified beforehand.

In some example embodiments, the encoder 113 may generate the representation 156 of the input protein molecule 150 by at least applying a structural role based numbering scheme in which each amino acid residue in the input sequence 152 is assigned an integer corresponding to a structural role of the amino acid residue. For example, in instances where the input sequence 152 corresponds to an immunoglobulin protein molecule (or antibody), each structural role may correspond to a different location in a region of the immunoglobulin protein such as a particular complementarity determining region (CDR) loop, a framework region between a pair of complementarity determining region (CDR) loops, and/or the like. Accordingly, the integer assigned to an amino acid residue in the input sequence 152 and the corresponding spot the amino acid residue occupies in the representation 156 of the input sequence 152 may be indicative of the structural role of the amino acid residue.

In some cases, the structural role based numbering scheme may include one or more structural roles that are not present in every protein sequence. Thus, it should be appreciated that a gap may be present in the representation 156 of the input sequence 152 at any spot in the input sequence 152 where an amino acid residue having the corresponding structural role is lacking. In some cases, the representation 156 of the input sequence 152 may be in a table or a matrix. For example, in some cases, the quantity of rows in the representation 156 may correspond to the quantity of structural roles in the structural based numbering scheme applied to generate the representation 156 of the input sequence 152. Moreover, in some cases, the quantity of columns in the representation 156 of the input protein molecule 150 may also be fixed. For instance, in some cases, the representation 156 may include one column populated by the type (or identity) of the amino acid residue (e.g., one-hot encoding of the residue type) occupying each row. Furthermore, the remaining columns in the representation 156 may be populated by the positions of the different possible atoms (e.g., a carbon, β carbon, oxygen, nitrogen, and/or the like) that can form the amino acid residue occupying each row. In some cases, as the input protein molecule 150 may occupy a multidimensional space, the position of each atom may be defined by multidimensional coordinates (e.g., three-dimensional Cartesian coordinates (x, y, z)). Thus, in some cases, the representation 156 of the input protein molecule 150 may include multiple channels, each of which corresponding to one dimension of the multidimensional coordinates defining the positions of the atoms.

Accordingly, in some cases, each row in the representation 156 (e.g., table, matrix, and/or the like) may correspond to a particular structural role in the structural based numbering scheme. For each structural role, the representation 156 of the input sequence 152 may include the type of amino acid residue at the corresponding row if in the input sequence 152 has an amino acid residue with the structural role. Alternatively, if the input sequence 152 lacks an amino acid residue having a certain structural role, the corresponding row in the representation 156 may be occupied by a gap character (or a “ghost residue”) to indicate the absence of an amino acid residue having the structural role. In some cases, each row in the representation 156 of the input sequence 152 may include an identifier of the type of amino acid residue or gap character occupying the row. Furthermore, each row of the representation 156 may include the positions (e.g., Cartesian coordinates (x, y, z)) of at least some of the atoms (e.g., heavy atoms) in the amino acid residue having the corresponding structural role. As described in more details below, the positions (e.g., Cartesian coordinates (x, y, z)) of a nonexistent atom, whether the atom forms an existent amino acid residue or a ghost residue at various gaps in the input sequence 152 may be determined based on the positions of the nearest existent atoms.

At 206, the protein design engine 110 may apply the protein design computation model 115 to generate, based at least on the representation of the input protein molecule, an output protein molecule by at least jointly denoising of the input sequence and the input three-dimensional structure of the input protein molecule to generate an output sequence and an output three-dimensional structure of output the protein molecule. In some example embodiments, the protein design engine 110 may apply the protein design computation model 115 to generate the protein molecule 160 by jointly co-designing the output sequence 162 and the output three dimensional structure 164 of the output protein molecule 160. In some cases, the protein design computation model 115 may generate the output sequence 162 and the output three-dimensional structure 164 of the output protein molecule 160 by jointly denoising the input sequence 162 and the input three-dimensional structure 164. The joint denoising may include updating the input sequence 152 and the input three-dimensional structure 154 to determine the discrete residue types forming the output sequence 162 as well as the continuous positions (e.g., Cartesian coordinates (x, y, z)) of the atoms forming each amino acid residue in the output three-dimensional structure 164. As described in more details below, in some cases, the protein design computation model 115 may be a diffusion model that jointly denoises the input sequence 152 and the input three-dimensional structure 154 over successive steps, with a portion of the noise present in the input protein molecule 150 being removed by the incremental updating of the input sequence 152 and the input three-dimensional structure 154 at each step.

In some example embodiments, the protein design computation model 115 may perform an invariant diffusion of the types of amino acid residues present in the input sequence 152 such that the types of amino acid residues identified by the protein design computation model 115 as present in the output sequence 162 are unaffected by the Euclidean transformations (e.g., rotations, translations, reflections, and/or the like) present in the input three-dimensional structure 154. Furthermore, the protein design computation model 115 may perform an equivariant diffusion of positions of the atoms in the input three-dimensional structure 154 such that the output three-dimensional structure 164 exhibits the same Euclidean transformations (e.g., rotations, translations, and/or the like) as the input three-dimensional structure 154.

In some example embodiments, the protein design computation model 115 may achieve equivariance to rotations through frame averaging in which the protein design computation 115 operates on four different frames at each step of the generative diffusion process. Each of the four frames may correspond to the input three-dimensional structure 154 being oriented in one of two possible directions along two out of the three principal axes of rotation about the centroid of the input three-dimensional structure 154. In this context, frame averaging refers to the output of the protein design computation model 115 at each step being an average of the output of the protein design computation model 115 operating on each of the four frames. For example, in some cases, the output of the protein design computation model 115 at each step may be an average of the protein design computation model 115 denoising a first frame in which the input three-dimensional structure 154 is oriented in one direction along a first principal axis of rotation, a second frame in which the input three-dimensional structure 154 is oriented in the opposite direction along the first principal axis of rotation, a third frame in which the input three-dimensional structure 154 is oriented in one direction along a second principal axis of rotation, and a fourth frame in which the input three-dimensional structure 154 is oriented in the opposite direction along the second principal axis of rotation, It should be appreciated that frame averaging may exclude the third principal axis of rotation in order to respect the fixed chirality of the input three-dimensional structure 154. That is, the input three-dimensional structure 154 may have superimposable mirror images along two of the three principal axes of rotation, meaning that the shape of the mirror image of the input three-dimensional structure 154 oriented in opposite directions along these two principal axes of rotation are identical. However, the shape of input three-dimensional structure 154 oriented in one direction along the third principal axis of rotation may be non-superimposable (or non-identical) with respect to its mirror image oriented in the opposite direction along the third principal axis of rotation.

In some example embodiments, in order to achieve equivariance to translations, the protein design computation model 115 may adjust a center of mass of the input three-dimensional structure 154 prior and subsequent to the joint denoising. In some cases, the center of mass of the input three-dimensional structure 154 may correspond to a mean (or average) of the positions of the atoms forming the input three-dimensional structure 154. Moreover, in some cases, the protein design computation model 115 may adjust the center of mass of the input three-dimensional structure 154 by at least subtracting the center of mass prior to the joint denoising before adding the center of mass back subsequent to the joint denoising. Doing so may correspond to translating the center of mass of the input three-dimensional structure 154 to certain Cartesian coordinates (e.g., (0, 0, 0)) before the joint denoising and translating the center of mass of the input three-dimensional structure 154 back to its original Cartesian coordinates after the joint denoising.

FIG. 2B depicts a flowchart illustrating an example of a process 250 for protein design, in accordance with some example embodiments. Referring to FIGS. 1 and 2A-B, the process 250 may be performed by the protein design engine 110, for example, the protein design computation model 115, to generate the protein molecule 160 by denoising the input sequence 152 and the input three-dimensional structure 154 of the input protein molecule 150. In some cases, the process 250 may implement operation 206 of the process 200 in which the protein design computation model 115 is applied to perform, over multiple successive steps, a joint denoising of the input sequence 152 and the input three-dimensional structure 154 to generate the output sequence 162 and the output three-dimensional structure 164 of the output protein molecule 160. Integrating protein sequence design and structural analysis through the joint denoising of the input sequence 152 and the input three-dimensional structure 154 may ensure that the output three-dimensional structure 164 assumed by the output sequence 162 are consistent with one or more desirable properties. For example, in some cases, the protein design computation model 115 may jointly denoise the input sequence 152 and the input three-dimensional structure 154 such that the output three-dimensional structure 164 assumed by the output sequence 162 complements the three-dimensional structure of a target molecule, such as a viral antigen, a tumor antigen, and/or the like. Moreover, as described in more details below, the protein design computation model 115 may integrate protein sequence design and structural analysis to explore sequence and conformational variations in a strategic and resource efficient manner, including by updating the positions of the atoms in the input three-dimensional structure 164 freely in three-dimensional space during the generative diffusion process while still imposing one or more bond constraints to ensure that the output three-dimensional structure 164 is consistent with those found in nature.

At 252, the protein design computation model 115 may modify, based at least on one or more bond constraints, an input three-dimensional structure of an input protein molecule having an input sequence and the input three dimensional structure. In some example embodiments, the protein design computation model 115 may impose one or more bond constraints to ensure that the output three-dimensional structure 164 of the output protein molecule 160 generated by the protein design computation model 115 is consistent with the protein molecules found in nature. For example, the one or more bond constraints may include idealized bond lengths and idealized bond angles. In some cases, the protein design computation model 115 may impose the one or more bond constraints by at least modifying the input three-dimensional structure 154 of the input protein molecule 150 before the input three-dimensional structure 154 undergoes joint diffusion along with the input sequence 152 of the input protein molecule 150. As will be explained in more details below, the one or more bond constraints may also be imposed on the output three-dimensional structure 164 of the output protein molecule 160 by at least modifying the updated input three-dimensional structure 154 that is generated by the joint diffusion.

As noted, in some cases, the protein design computation model 115 may be a diffusion model implemented using one or more artificial neural networks (ANNs) such as multilayer perceptrons (MLPs) and/or the like. In some cases, the protein design computation model 115 may include one or more projection layers for imposing the one or more bond constraints on the input three-dimensional structure 154 and the target three-dimensional structure 164. For example, in some cases, the one or more projection layers may modify, based on the one or more bond constraints, the input three-dimensional structure 154 prior to the joint diffusion performed by the diffusion model (e.g., the one or more artificial neural networks (ANNs). Alternatively and/or additionally, the one or more projection layers may modify the updated input three-dimensional structure 154 generated by the joint diffusion such that the output three-dimensional structure 164 of the output protein molecule 160 are consistent with the one or more bond constraints.

In some example embodiments, the one or more projection layers may modify the input three-dimensional structure 154 (or the updated input three-dimensional structure 154) by operating on the representation 156 of the input protein molecule 150. As noted, the representation 156 may be a fixed-sized representation of the input protein molecule 150 that includes the actual positions (e.g., three-dimensional Cartesian coordinates (x, y, z)) of existent atoms (e.g., heavy atoms) as well as the interpolated positions (e.g., three-dimensional Cartesian coordinates (x, y, z)) of nonexistent atoms, whether those atoms are part of an existent amino acid residue or a nonexistent amino acid residue. For example, in the case of a nonexistent atom in an existent amino acid residue, the position (e.g., three-dimensional Cartesian coordinates (x, y, z)) of the nonexistent atom may be determined based on the positions (e.g., three-dimensional Cartesian coordinates (x, y, z)) of one or more existent atoms in the amino acid residue (e.g., one or more nearest atoms, one or more atoms within a threshold distance, and/or the like). In the case of a nonexistent amino acid residue represented by a gap character, the positions of the constituent atoms may be determined (e.g., interpolated, extrapolated, and/or the like) based on the positions of the atoms in one or more existent amino acid residues (e.g., one or more nearest amino acid residues in space, one or more adjacent amino acid residues in sequence, one or more amino acid residues within a threshold distance, and/or the like). Accordingly, in some cases, the one or more projection layers may modify the input three-dimensional structure 154 (or the updated input three-dimensional structure 154) by at least modifying the positions (e.g., three-dimensional Cartesian coordinates (x, y, z)) of the atoms included in the representation 156 of the input protein molecule 150.

In some example embodiments, the one or more projection layers may modify the input three-dimensional structure 154 (or the updated input three-dimensional structure 154) by at least aligning the input three-dimensional structure 154 to a reference residue backbone formed from a plurality of rigid atoms with fixed bond lengths and fixed bond angles (e.g., idealized bond lengths and idealized bond angles). For example, in some cases, the one or more projection layers may align the input three-dimensional structure 154 to the reference residue backbone by at least determining one or more transformations (e.g., roto-translation) that can be applied to the positions (e.g., Cartesian coordinates (x, y, z)) of the backbone atoms in the input three-dimensional structure 154 to minimize the distance (e.g., root mean squared deviation (RMSD)) between the atoms in the input three-dimensional structure 154 and the atoms in the reference residue backbone.

Alternatively and/or additionally, the one or more bond constraints may be imposed based on the side chain templates of the types of amino acid residue present in the input protein molecule 150. For example, in some cases, each type of amino acid residue may be associated with a different side chain template specifying the idealized dihedral angles (or torsion angles) formed by the constituent atoms. It should be appreciated that an amino acid residue may have up to four bonds, each of which rotating about a corresponding dihedral angle. However, the one or more projection layers may defer modifying the input three-dimensional structure 154 based on any specific side chain templates at least because the exact identity of the amino acid residues forming the input three-dimensional structure 154 will change during the generative diffusion process. Thus, instead of specific side chain templates, the one or more projection layers may first replace the side chains of the input three-dimensional structure 154 with a generic representation with sufficient degrees of freedom to account for the placement of the atoms in each of the side chains. For instance, the side chains of amino acid residues may include up to four bonds, each of which being rotatable about a corresponding dihedral angle (or torsion angle). Accordingly, the generic representation of the side chains may include four pseudo atoms (e.g., pseudo carbon atoms) having of the same rotational freedom (e.g., about the dihedral angles) as those in the original side chain. Where the original side chain lacks a degree of rotational freedom about a particular dihedral angle, that dihedral angle in the generic representation of the side chain may be set to a fixed angle (e.g., 180° and/or the like) in order to fix the atoms forming the dihedral angle along a plane. As described in more details below, the generic representations of the side chains in the updated input three-dimensional structure 154 may be replaced by the one or more projection layers when generating the output three-dimensional structure 164 of the output protein molecule 160.

At 254, the protein design computation model 115 may perform, over one or more successive steps, one or more incremental updates to the input protein molecule that each include a joint denoising of the input sequence and the input three-dimensional structure of the input protein molecule. In some example embodiments, the protein design computation model 115 may be a diffusion model that generates the output sequence 162 and the output three-dimensional structure 164 of the output protein molecule 160 by jointly denoising the input sequence 152 and the input three-dimensional structure 154 of the input protein molecule 150. It should be appreciated that the joint denoising of the input sequence 152 and the input three-dimensional structure 154 may be an iterative process that defines a gradual transition between includes a succession of incremental updates occurring over multiple steps. For example, the forward diffusion process may include the incremental addition of noise to a datapoint X₀ to form the corrupted datapoint X_{T} over multiple steps to form a trajectory of samples (Xₒ, X₁, . . . , Xₜ, . . . , X_{T}) containing incrementally greater quantities of noise. In some cases, the samples may interpolate from the data distribution X₀˜p(X₀) to that of an easy-to-sample prior data distribution X_{T}˜p(X_{T}), such as a Gaussian distribution and/or the like. Moreover, in some cases, the forward diffusion process may be Markovian in nature, meaning that

$$q ( {X}_{0} ) = q ( {X}_{1} \vert {X}_{0} ) {\prod }_{t = 2}^{T} q ( {X}_{t} \vert {X}_{t - 1} ) .$$

It should be appreciated that the protein design computation model 115 may be trained to learn a denoising process \hat{X}₀=ϕ(Xₜ, t) for recovering the datapoint X₀ from the corrupted datapoint X_{T}. For example, in some cases, the denoising process \hat{X}₀ may be learned through minimizing the variational upper bound on the negative loglikelihood, which in the context of protein design may mean approximating the data distribution of known protein molecules (or a certain subset thereof) to increase (or maximize) the likelihood of the output protein molecule 160 being within the higher density regions of that data distribution Moreover, the aforementioned generative diffusion process may include the protein design computation model 115 being applied to generate, from the corrupted datapoint X_{T}, the datapoint X₀ over multiple successive iterations (X_{T}>X_{T-1}> . . . >X₁>X₀).

In some example embodiments, the protein design computation model 115 may jointly denoise the input sequence 152 and the input three-dimensional structure 154 by at least performing joint modifications to the types of amino acid residues in the input sequence 152 and the positions (e.g., Cartesian coordinates (x, y, z)) of the constituent atoms. For example, in some cases, the diffusion process may factorize the posterior probability distribution over atomic positions as well as the type of amino acid residues:

$$q ( {X}_{t - 1} ) = q ( {X}_{t - 1}^{pos} ) q ( {X}_{t - 1}^{res} )$$

wherein a Gaussian distribution may govern the noise applied to atomic positions while a categorical distribution may govern the noise applied to the types of amino acid residues. The reverse diffusion process, which generates the output protein molecule 160 by jointly modifying the input sequence 152 and the input three-dimensional structure 154, may operate on the representation 156 of the input protein molecule 150. Since the reverse diffusion process to generate the output sequence 162 and the output three-dimensional structure 164 is joint, the protein design computation model 115 may jointly predict atomic positions and residue types

$$( {\hat{X}}_{0}^{pos} , {\hat{X}}_{0}^{res} ) = ϕ ( {X}_{t}^{pos} , {X}_{t}^{res} , t ) ,$$

wherein X^{pos}∈R^{n×3} is a matrix of atomic positions (e.g., Cartesian coordinates (x, y, z)) and X^{res}∈R^{m×21} is a matrix of encoded residue types (e.g., one-hot encoding of 20 canonical amino acid residues and a gap character). For example, in some cases, the joint denoising may include performing, at each step, an incremental update to the type of amino acid residue (e.g., one-hot encoding of the residue type) in the input sequence 152 and/or the positions (e.g., Cartesian coordinates (x, y, z)) of the atoms in each amino acid residue. Each incremental update may remove a portion of the noise that is present in the input protein molecule 150 to gradually increase (or maximize) the likelihood of the output protein molecule 160 being in a higher density region of the data distribution of known protein molecules (or a certain subset thereof).

The ability to efficiently sample any step in the forward diffusion process

$$q ( {X}_{t}^{pos} \vert {X}_{0}^{pos} ) ,$$

an important feature in the training of the protein design computation model 115, may be achieved using a Gaussian distribution

$$q ( {X}_{0}^{p o s} ) = N ( {α}_{t} {X}_{0}^{pos} , {σ}_{t}^{2} Σ ) ,$$

wherein αₜ and

$${σ}_{t}^{2}$$

are positive, scalar-valued functions of t, with α₁≈1 and α_{T}≈0. One way to define the noising process is the variance-preserving parametrization with

$${α}_{t} = \sqrt{1 - {σ}_{t}^{2} \cdot } .$$

A single forward diffusion step in this general diffusion formulation may be expressed as

$$\begin{matrix}q ( {X}_{t - 1}^{pos} ) = N ( {α}_{t \vert t - 1} {X}_{0}^{pos} , {σ}_{t \vert t - 1}^{2} Σ ) , wherein {α}_{t \vert t - 1} = \frac{{α}_{t}}{{α}_{t - 1}} and \\ {σ}_{t \vert t - 1}^{2} = {σ}_{t}^{2} - {α}_{t \vert t - 1}^{2} {σ}_{t \vert t - 1}^{2} .\end{matrix}$$

Similarly, a single reverse diffusion step

$$q ( {X}_{t}^{pos} , {X}_{0}^{pos} )$$

can then be expressed as the following Gaussian

$$N ( \frac{{α}_{t \vert t - 1} {σ}_{t - 1}^{2} {X}_{t}^{pos} + {α}_{t - 1} {σ}_{t \vert t - 1}^{2} {X}_{0}^{pos}}{{σ}_{t}^{2}} , \frac{{σ}_{t \vert t - 1} {σ}_{t - 1}}{{σ}_{t}} Σ ) .$$

The training objective for the Gaussian distribution can be simplified to

$${L}_{t} ( {X}_{0}^{pos} ) = \frac{1}{2} ( SNR ( t - 1 ) - SNR ( t ) ) {‖ {\hat{X}}_{0}^{pos} - {X}_{0}^{pos} ‖}_{{Σ}^{- 1}}^{2} ,$$

wherein the signal-to-noise ratio is

$$SNR ( t ) = {( \frac{{α}_{t}}{{σ}_{t}} )}^{2} and {‖ \cdot ‖}_{{Σ}^{- 1}}^{2} = {( \cdot )}^{T} {Σ}^{- 1} ( \cdot ) .$$

In some cases, predicting the noise

$$\hat{ϵ} = ϕ ( {X}_{t}^{p o s} , t )$$

that has been added

$${X}_{t}^{p o s} = {α}_{t} {X}_{0}^{p o s} + {σ}_{t} ϵ , ϵ ∼ N ( 0 , \sum )$$

and using an unweighted loss

$${L}_{t} ( {X}_{0}^{p o s} ) = \frac{1}{2} {‖ \hat{ϵ} - ϵ ‖}_{{\sum }^{- 1}}^{2}$$

may improve model training because this parameterization makes it easier for the model to minimize the loss at time steps close to T. Alternatively, a similar effect may be achieved by re-weighing the mean prediction loss

$${L}_{t} ( {X}_{0}^{p o s} ) = \frac{1}{2} S N R ( t ) {‖ {\hat{X}}_{0}^{p o s} - {X}_{0}^{p o x} ‖}_{{\sum }^{- 1}}^{2} ,$$

which empirically renders the mean

$${\hat{X}}_{0}^{p o s} = ϕ ( {X}_{t}^{p o s} , t ) and error \hat{ϵ} = ϕ ( {X}_{t}^{p o s} , t )$$

prediction alternatives perform comparably. Predicting

$${\hat{X}}_{0}^{p o s} = ϕ ( {X}_{t}^{p o s} , t )$$

may also permit for easier incorporation of known constraints, such as atom bond lengths. Moreover, it should be appreciated that

$${L}_{1} ( {X}_{0}^{p o s} )$$

can also be optimized using the same objective.

In some cases, the discrete diffusion process for the residue types may be formulated as a discrete random variable with k categories, represented by a one-hot vector X^{res}. The forward diffusion process may be defined as

$$q ( {X}_{0}^{r e s} ) = Cat ( {X}_{0}^{r e s} {Q}_{t} ) ,$$

where the transition matrix Qₜ=βₜI+(1−βₜ) Q is a linear interpolation between identity and target distribution Q (e.g., a uniform distribution). Similarly to the atom diffusion, a single forward diffusion step may be expressed as

$$q ( {X}_{t - 1}^{r e s} ) = C a t ( {X}_{t}^{r e s} {Q}_{t \vert t - 1} ) , wherein {Q}_{t \vert t - 1} = {Q}_{t - 1}^{- 1} {Q}_{t} .$$

From this, a single reverse diffusion step may be expressed as

$$q ( {X}_{t}^{r e s} , {X}_{0}^{r e s} ) = Cat ( \frac{{X}_{t}^{r e s} {Q}_{t \vert t - 1}^{T} ⊙ {X}_{0}^{res} {Q}_{t - 1}}{Z} ) ,$$

for an appropriate normalizing constant Z. Similarly to the case of atom position diffusion, a simplified objective,

$${L}_{t} ( {X}_{0}^{r e s} ) = - {β}_{t} log log {p}_{θ} ( {X}_{0}^{r e s} \vert {X}_{t}^{r e s} ) ,$$

may be used for the discrete diffusion of residue types to achieve better results. Hyperparameter βₜ may ensure that the loss weight is proportional to the fraction of unchanged classes. This hyperparameter may be set as

$${β}_{t} = {α}_{t}^{2}$$

to keep the noise schedules for the residue type diffusion and atom position diffusion the same. It should be appreciated that the L₁ (X₀) may simply be the cross entropy of the model prediction L₁ (X₀)=log log p_{θ}(X₀|X₁).

At 256, the protein design computation model 115 may modify, based at least on the one or more bond constraints, the updated three-dimensional structure of the input protein molecule. In some example embodiments, the protein design computation model 115 may modify, based at least on the one or more bond constraints, the updated input three-dimensional structure 154 of the input protein molecule 150 generated by the joint denoising. As noted, in some cases, the joint denoising of the input sequence 152 and the input three-dimensional structure 154 may include the protein design computation model 115 freely moving, for example, in three-dimensional space, the positions of the atoms forming the input protein molecule 150. Doing so may introduce artifacts including, for example, unrealistic bond lengths, unrealistic bond angles, and/or the like. In some cases, the protein design computation model 115, for example, the one or more projection layers, may modify the updated input three-dimensional structure 164 in order to remove the artifacts introduced during the joint denoising of the input sequence 152 and the input three-dimensional structure 154.

In some example embodiments, the one or more projection layers of the protein design computation model 115 may modify the updated input three-dimensional structure 164 by at least aligning the updated input three-dimensional structure 164 to a reference residue backbone formed from a plurality of rigid atoms with fixed bond lengths and fixed bond angles (e.g., idealized bone lengths and idealized bond angles). For example, in some cases, the one or more projection layers may align the updated input three-dimensional structure 154 to the reference residue backbone by at least applying, to the positions (e.g., Cartesian coordinates (x, y, z)) of the constituent atoms, one or more transformations (e.g., roto-translation) that minimizes the distance (e.g., root mean squared deviation (RMSD)) between the atoms in the updated input three-dimensional structure 154 and the atoms in the reference residue backbone. Alternatively and/or additionally, the updated input three-dimensional structure 154 may be further modified by replacing the generic side chains in the updated input three-dimensional structure 154 with the side chain templates of the specific types of amino acid residues identified as forming the output protein molecule 160. As noted, the types (or identities) of the amino acid residues forming the output protein molecule 160 may be indeterminate throughout the generative diffusion process, which is why joint diffusion may be performed on generic representation of the side chains, which includes pseudo atoms (e.g., pseudo carbon atoms) having the same degrees of freedom instead of the side chain templates of specific types of amino acid residues. Once the types (or identities) of the amino acid residue forming the output protein molecule 160 are certain, the one or more projection layers may modify the bond length between the pseudo atoms (e.g., 1.54 Angstroms for carbon atoms). Furthermore, the generic representations of the side chains in the updated input three-dimensional structure 154 may be replaced with the corresponding side chain templates, which includes replacing the pseudo atoms with the actual atoms present in each side chain template and applying the dihedral angles therebetween.

At 258, the protein design computation model 115 may generate an output protein molecule to include the updated input sequence and the modified input three-dimensional structure. In some example embodiments, the protein design computation model 115 may generate the output protein molecule 160 to include the updated input sequence 154 as the output sequence 162 and the modified input three-dimensional structure 154 as the output three-dimensional structure 164. Moreover, the protein design computation model 115 may generate the output protein molecule 160 for a variety of applications. For example, where the input sequence 152 and/or input three-dimensional structure 154 is associated with another protein molecule from a particular family of protein molecules (e.g., immunoglobulins (or antibodies) and/or the like), the protein design computation model 115 may generate the output protein molecule 160 to be additional member of the same protein family. Alternatively and/or additionally, the input sequence 152 and/or input three-dimensional structure 154 may be associated with another protein molecule exhibiting one or more desirable properties, such as binding affinity towards a target molecule (e.g., a viral antigen, a tumor antigen, and/or the like). Accordingly, the protein design computation model 115 may generate the output protein molecule 160 to exhibit the same desirable properties or, in some cases, one or more additional desirable properties such as expression, specificity, stability, immunogenicity, human-ness, lack of self-association, and/or the like.

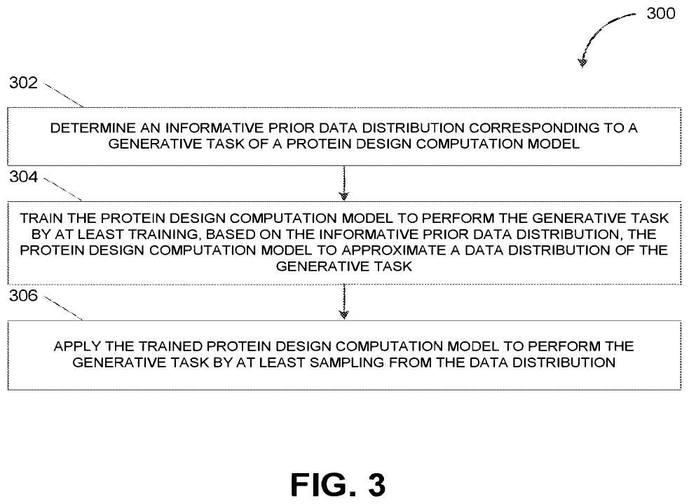

FIG. 3A depicts a flowchart illustrating another example of a process 300 for protein design, in accordance with some example embodiments. Referring to FIGS. 1 and 3A, the process 300 may be performed by the protein design engine 110 to incorporate an informative prior into the generative diffusion process. For example, in some cases, the protein design engine 110 may train the protein design computation model 150 to perform a generative task such as the joint diffusion of the input sequence 152 and the input three-dimensional structure 154 of the input protein molecule 150 to generate the output sequence 162 and the output three-dimensional structure 164 of the output protein molecule 160. In some cases, the protein design computation model 150 may perform the generative task by at least sampling from a data distribution learned through training. As described in more details below, the protein design computation model 115 may be trained based on an informative prior data distribution that is associated with the generative task. That is, in some cases, the protein design computation model 115 may learn to approximate the posterior data distribution (or true data distribution) of protein molecules based on a prior data distribution that reflects at least some characteristics of protein molecules (or certain subsets of protein molecules). For instance, where the generative task includes generating immunoglobulin protein molecules (or antibodies), the informative prior data distribution may capture certain characteristics of immunoglobulin protein molecules (or antibodies) such as the types of amino acid residues and/or positions of the atoms typically observed in immunoglobulin protein molecules (or antibodies). Incorporating the informative prior data distribution may reduce the complexity of the protein design computation model 115 needed to achieve good-quality denoising, expedite the generation of the output protein molecule 160, and improve data efficiency.

At 302, the protein design engine 110 may determine an informative prior data distribution corresponding to a generative task of the protein design computation model 115. In some example embodiments, the protein design engine 110 may determine an informative prior data distribution that is specific to the generative task of the protein design computational model 115. Unlike a noninformative prior data distribution (e.g., normal (Gaussian) distribution, uniform distribution, and/or the like) that is agnostic to the type of protein molecule the protein design computation model 115 is being trained to generate, the informative prior data distribution may reflect at least some of the characteristics of these protein molecules. For example, in cases where the protein design computation model 115 is trained to generate protein molecules from a specific protein family (e.g., immunoglobulin proteins (or antibodies)), the protein design computation model 115 may be trained to learn, based on an informative prior data distribution associated with that protein family, a posterior data distribution from which to sample from during the generative diffusion process. In some cases, the protein design engine 110 may determine the informative prior data distribution to be similar to the posterior data distribution approximated by the trained protein design engine 110. As described in more details below, the informative prior data distribution may determine the noise that is added to generate the corrupted datapoint X_{T} at every step in the forward diffusion process.

In the context of protein generation, the informative prior data distribution may reflect at least some characteristics of the types of protein molecule (e.g., immunoglobulin protein molecules (or antibodies), protein molecules with certain properties, and/or the like) the protein design computation model 115 is being trained to generate. For instance, in some cases, the protein design engine 110 may generate the informative prior data distribution to have a small distance (e.g., Wasserstein distance) relative to the posterior data distribution. Where the generative process includes a joint diffusion of residue type and atomic positions, the informative prior data distribution may capture the positional amino-acid residue frequency and/or conditional atom dependencies observed in certain protein molecules.

To further illustrate, consider the posterior data distribution p and the learned data distribution p_{θ}, the latter being obtained by the push-forward p_{θ}(ƒ_{θ}(Z))=q(Z) updating some prior data distribution q by the function ƒ_{θ}. In the context of denoising diffusion, the reverse diffusion process learned by the protein design computation model 115 may be expressed as Z=X_{T} with the function ƒ_{θ} being the denoising function (e.g., ƒ_{θ}=ϕ(X_{T}, T)). It should be appreciated that the quality of the protein design computation model 115 as a generative model and the complexity of the aforementioned denoising function ƒ_{θ} may be dependent on the informativeness of the prior data distribution q. That is, for any denoising function ƒ_{θ} that is an invertible equivariant function with respect to a subgroup G of the general linear group GL(d, [symbol] ), the complexity measure c_{q} of the protein design computation model 115 may be subject to the following bound:

$${c}_{q} ( {f}_{θ} ) \geq {W}_{t} ( p , q ) - {W}_{t} ( p , {p}_{θ} ) , wherein$$

$${c}_{q} ( f ) := {( {min}_{g ∈ G} {E}_{Z ∼ q} [ {‖ f ( gZ ) - Z ‖}_{t}^{t} ] )}^{\frac{1}{t}}$$

quantifies the expected complexity of the protein design computation model 115 under the prior data distribution q, Wₜ (p, p_{θ}) is the Wasserstein t-distance between the learned distribution p_{θ} and the posterior data distribution p, and Wₜ (p, q) is the Wasserstein distance between the prior data distribution q and the posterior data distribution p.

This bound indicates that a simple generative model (in terms of the complexity measure c_{q} (ƒ)) that also fits the data well cannot be learned unless the prior data distribution q has a small distance (e.g., Wasserstein distance) to the posterior data distribution p. According to the following equation, in the context of denoising diffusion, the complexity measure c_{q} may correspond to the expected amount of denoising the protein design computation model 115 performs throughout the reverse diffusion process, discounting for rotations. Increasing the informativeness of the prior data distribution q may reduce the amount of denoising performed by the protein design computation model 115 when generating the output protein molecule 160.

$${c}_{q} ( ϕ ) = {min}_{g ∈ S E ( 3 )} {E}_{{X}_{T}} [ {‖ ϕ ( g {X}_{T} , T ) - {X}_{T} ‖}_{2}^{2} ]$$

As noted, in some example embodiments, the informative prior data distribution may reflect at least some characteristics of the characteristics of the protein molecules the protein design computation model 115 is being trained to generate. Moreover, in some cases, the informative prior data distribution may be generated based on known protein sequences (e.g., the Observed Antibody Space (OAS), the Protein Data Bank (PDB), and/or the like) or a certain subset thereof (e.g., protein molecules exhibiting one or more desirable properties). For example, in some cases, the informative prior data distribution may be generated based on a training set of protein sequences to include the frequency at which different types of amino acid residues (e.g., of the twenty canonical amino acid residues) appear at least some of the positions within the protein sequences in the training set. In instances where each protein sequence in the training set is rendered in a fixed-sized representation that is generated by applying a structural role based numbering scheme, the informative prior data distribution may indicate the frequency at which different types of amino acid residues assume at least some of the possible structural roles including, in some cases, the frequency of gaps where at least some structural roles are vacant instead of being filled by an amino acid residue. That is, the informative prior data distribution may account for the frequency of gaps where no amino acid residue assumes a particular structural role.

In some cases, the informative prior data distribution containing the marginal position-specific categorical distributions over residues types (e.g., Q¹, . . . , Q^{2*149}) may be incorporated during the forward diffusion process in which the corrupted datapoint X_{T} is generated with the incremental addition of noise over successive steps. That is, instead of the corrupted datapoint X_{T} being generated to exhibit noise sampled from a noninformative prior data distribution, such as a uniform probability distribution across the possible residue types where each residue type is equally likely, the corrupted datapoint X_{T} may be generated to capture, based on the informative prior data distribution, the observation that a first residue type is more likely to occupy certain positions in a protein sequence (e.g., an immunoglobulin protein sequence (or an antibody)) than a second residue type. It should be appreciated that the informative prior data distribution may conform with empirical observations such as, for example, that the estimated prior data distributions of residue identity in at least some protein molecules, such as immunoglobulin protein molecules (or antibodies), are far from uniform. Instead, some portions of a protein molecule, such as the preserved regions of the immunoglobulin fold, may exhibit especially low entropy. Furthermore, in some positions within a protein molecule, such as the usually shorter light chain, certain types of amino acid residues may be observed across the most if not all of the known protein sequences (e.g., the entirety of the Observed Antibody Space (OAS)), meaning that these positions may be excluded altogether. As described in more details below, the informative prior data distribution capturing positional amino acid residue frequency may be incorporated into the training of the protein design computation model 115 by at least adjusting, in accordance to the residue frequency, the noise

$$q ( {X}_{t}^{r e s} \vert {X}_{t - 1}^{r e s} ) = C a t ( {X}_{t}^{r e s} ( {β}_{t} I + ( 1 - {β}_{t} ) {Q}^{i} ) )$$

that is introduced to residue type (or identity) at every step of the forward diffusion process, which the protein design computation model 115 then learns to remove as part of the reverse diffusion process. In particular, the noise

$$q ( {X}_{t}^{r e s} \vert {X}_{t - 1}^{r e s} )$$

may be position-specific, meaning that the noise

$$q ( {X}_{t}^{r e s} \vert {X}_{t - 1}^{r e s} )$$

depends on the residue position i each protein sequence.

Alternatively and/or additionally, the informative prior data distribution may capture the conditional dependencies that exist in the position of the atoms in the amino acid residues forming a protein molecule. For example, where two (or more) amino acid residues are adjacent to one another in a protein sequence, the chain-like conformation of protein molecules means that the constituent atoms are more likely to occupy proximate positions in the corresponding three-dimensional structure as well. That is, protein molecules in nature assume a chain-like conformation, which limits the extent to which the positions of some atoms can change relative to that of other atoms in adjacent amino acid residues. Changes in atomic positions that result in an excessive distance between the atoms in adjacent amino acid residues may disrupt the chain-like conformation and yield unrealistic three-dimensional structures. Accordingly, in some cases, the protein design engine 110 may generate, based on the three-dimensional structure of known protein molecules, an adjacency matrix specifying the conditional dependencies (and independencies) that exist between the atomic positions of neighboring amino acid residues. When the training of the protein design computation model 115 incorporates the conditional dependencies specified in the adjacency matrix, the protein design computation model 115 may be able to generate the output three-dimensional structure 164 to exhibit a more realistic chain-like conformation faster (e.g., over fewer denoising steps) and with greater computational efficiency.

At 304, the protein design engine 110 may train the protein design computation model 115 to perform the generative task by at least training, based on the informative prior data distribution, the protein design computation model 115 to approximate a data distribution of the generative task. In some example embodiments, the protein design computation model 115 may incorporate an informative prior data distribution, such as residue type frequency and conditional atom dependencies, into the forward diffusion process by at least of the protein design computation model 115. As noted, in a denoising diffusion framework, the protein design computation model 115 may perform a forward diffusion process that includes the gradual addition of noise to the data point X₀ to form the corrupted datapoint X_{T} over a trajectory (X₀, X₁, . . . , Xₜ, . . . , X_{T}) of increasingly noisy data. In some cases, incorporating the informative prior may include incorporating the informative prior data distribution, such as residue type frequency, conditional atom dependencies, and/or the like, into the aforementioned forward diffusion process. For example, in some cases, the informative prior data distribution may be incorporated into the forward diffusion process by adding, to the data point X₀, noise sampled from the informative prior data distribution instead noise sampled from a noninformative prior data distribution, such as a uniform distribution, a Gaussian (or normal) distribution, and/or the like, that is agnostic to the type of protein molecule the protein design computation model 115 is being trained to generate. Corrupting to the data point X₀ with noise sampled from a noninformative prior data distribution means that the corrupted data point X_{T} may exhibit unrealistic modifications (e.g., in residue types, atomic positions, and/or the like) that are unlikely (or even impossible) for the type of protein molecules the protein design computation model 115 is being trained to generate. Training the protein design computation model 115 to denoise the corrupted data point X_{T} by recognizing and removing unrealistic modifications may increase the complexity of the protein design computation model 115 without necessarily improving the quality of the denoising performed by the protein design computation model 115 as part of the generative task. Accordingly, incorporating an informative prior data distribution instead of a noninformative prior data distribution may ease the complexity of the reverse diffusion process (or denoising process) such that the protein design computation model 115 is able to generate the output protein molecule 160 faster (e.g., over fewer denoising steps) and with greater computational efficiency.

At 306, the protein design engine 110 may apply the trained protein design computation model 115 to perform the generative task by at least sampling from the data distribution. In some example embodiments, once trained, the protein design engine 110 may apply the protein design computation model 115 to generate the output protein molecule 160 by at least jointly denoising the input sequence 152 and the input three-dimensional structure 154 of the input protein molecule 150 to generate the output sequence 162 and the output three-dimensional structure 164 of the output protein molecule 160. In some cases, the joint denoising may be a part of the reverse diffusion process the protein design computation model 115 was trained to perform in operation 304. Moreover, in some cases, the protein design computation model 115, whose training incorporates an informative prior data distribution, may be applied to perform operation 206 of the process 200 shown in FIG. 2A as well as operation 254 of the process 250 shown in FIG. 2B. As noted, when the training of the protein design computation model 115 incorporates an informative prior data distribution instead of a noninformative prior data distribution, the protein design computation model 115 may generate the output protein molecule 160 faster (e.g., over fewer denoising steps) and with greater computational efficiency at least because the incorporation of the informative prior data distribution reduces the complexity of the reverse diffusion process. For example, in some cases, the protein design computation model 115 may jointly denoise joint the input sequence 152 and the input three-dimensional structure by at least incrementally updating, over multiple successive steps, the input sequence 152 and the input three-dimensional structure 154 to determine the discrete residue types forming the output sequence 162 as well as the continuous positions (e.g., Cartesian coordinates (x, y, z)) of the atoms forming each amino acid residue in the output three-dimensional structure 164.

In some example embodiments, the joint denoising of the input sequence 152 and the input three-dimensional structure 154 may follow a noise schedule defining the distribution of noise levels across a certain quantity of denoising steps. For example, in some cases, the joint denoising of the input sequence 152 and the input three-dimensional structure 154 may follow the cosine-like noise schedule shown below. In some cases, the noise schedule, such as the cosine-like noise schedule below, may define the distribution of noise levels across 1000 denoising steps:

$${α}_{t} = ( 1 - 2 s ) \cdot ( 1 - {( \frac{t}{T} )}^{2} ) + s ,$$

wherein a precision of s=1e⁻⁴ is used to avoid numerical instability, or calculations that can magnify approximation errors. As

$${σ}_{t} = \sqrt{1 - {α}_{t}^{2}} and {β}_{t} = {α}_{t}^{2} ,$$

it may be sufficient to define just αₜ. Furthermore, in order to avoid instability during sampling, the distribution of noise levels α_{t|t-1} across successive denoising steps t and t−1 may be clipped to at least 0.001 and αₜ may be recomputed as a cumulative product. In some cases, numerical stability may be further ensured by computing the negative log signal-to-noise (SNR) curve

$$γ ( t ) = - log {α}_{t}^{2} + log {σ}_{t}^{2} .$$

This parameterization may allow for numerically stable computation of various critical values including, for example,

$${α}_{t}^{2} = sigmoid ( - γ ( t ) ) , {α}_{t}^{2} = sigmoid ( γ ( t ) ) , {α}_{t \vert t - 1}^{2} = - expm 1 ( softplus ( γ ( t - 1 ) ) - softplus ( γ ( t ) ) = - expm 1 ( γ ( t - 1 ) - γ ( t ) ) {σ}_{t}^{2} , and S N R = exp ( - γ ( t ) ) , wherein expm 1 ( x ) = exp ( x ) - 1 and softplus 1 ( x ) = log ( 1 + exp ( x ) ) .$$

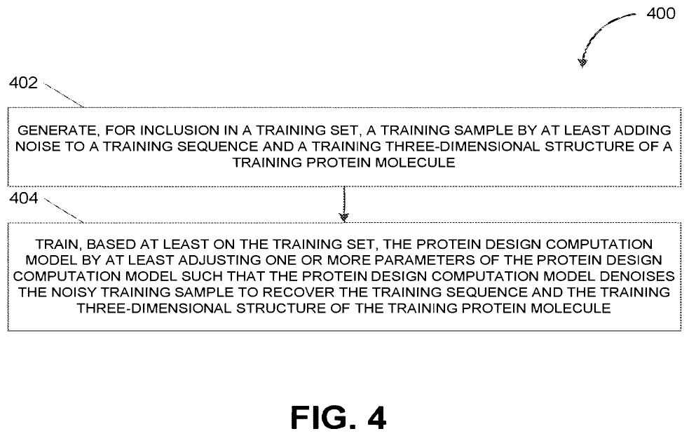

FIG. 4 depicts a flowchart illustrating an example of a process 400 for training the protein design computation model 115, in accordance with some example embodiments. Referring to FIGS. 1, 2A-B, 3A-B, and 4, the process 400 may be performed by the protein design engine 110 to train the protein design computation model 115 to perform a generative task that includes the joint denoising of the input sequence 152 and the input three-dimensional structure 154 of the input protein molecule 150 to generate the output sequence 162 and the output three-dimensional structure 164 of the output protein molecule 160. In some cases, the process 400 may implement operation 304 of the process 300 shown in FIG. 3A in which the training of the protein design computation model 115 incorporates an informative prior data distribution (e.g., positional amino acid residue frequency, conditional atom dependencies, and/or the like). However, it should be appreciated that the process 400 may also be performed to train the protein design computation model 115 without an informative prior data distribution. As described in more details below, the training of the protein design computation model 115 may include adjusting one or more parameters (e.g., weights, biases, and/or the like) of the protein design computation model 115 such that the protein design computation model 115 is able to denoise, incrementally over successive steps,

At 402, the protein design engine 110 may generate, for inclusion in a training set, a noisy training sample by at least adding noise to a training sequence and a training three-dimensional structure of a training protein molecule. In some example embodiments, the protein design engine 110 may generate, based on one or more training protein molecules, a training set for training the protein design computation model 115. In some cases, the one or more training protein molecules, each of which including a training sequence and a training three-dimensional structure, may be known protein molecules with known three-dimensional structure (e.g., from the Protein Data Bank (PDB)) or folded protein molecules (e.g., immunoglobulin protein molecules (or antibodies) from the Observed Antibody Space (OAS)). Moreover, the protein design engine 110 may generate, based on each training protein molecule, a corresponding noisy training sample for inclusion in the training set. For example, in some cases, the protein design engine 110 may generate a noisy training sample by at least adding noise to the training sequence and the training three-dimensional structure of a training protein molecule.

In some example embodiments, the forward diffusion process may be gradual, meaning that the noise is introduced incrementally over multiple successive steps. For example, in some cases, the protein design engine 110 may generate the noisy training sample by adding a first quantity of noise at a first step before a second quantity of noise is added at a second step. Moreover, in some cases, the forward diffusion process may include a joint noising in which both the training sequence and the training three-dimensional structure of each training protein molecule are modified with the addition of noise. For instance, in some cases, the protein design computation engine 110 may generate a noisy training sample by at least modifying the type (or identity) of one or more amino acid residues in the training sequence of a training protein molecule. In some cases, such modifications may include inserting an amino acid residue occupying a spot in the training sequence by replacing the gap character with the amino acid residue as well as deleting an amino acid residue at a spot in the training sequence by replacing the amino acid residue with a gap character. In addition to modifying the training sequence of the training protein molecule, the generating of the noisy training sample may also include the protein design engine 110 modifying the training three-dimensional structure of the training protein molecule by changing the positions (e.g., Cartesian coordinates (x, y, z)) of one or more atoms forming the training three-dimensional structure of the training protein molecule.

In instances where the training of the protein design computation model 115 incorporates an informative prior data distribution, such as positional amino acid residue frequency and/or conditional atom dependencies, the forward diffusion process may include the addition of noise sampled from the informative prior data distribution. As noted, the incorporation of the informative prior data distribution, which decreases the distance between the posterior data distribution and the one the protein design computation model 115 is being trained to approximate, may reduce the complexity of the trained protein design computation model 115. For example, where the informative prior data distribution includes the frequency with which different types of amino acid residues occupy each position in a protein sequence, the protein design engine 110 may generate a noisy training sample by at least modifying the training sequence of a training protein molecule in accordance with the aforementioned positional amino acid residue frequency. Thus, instead of the type (or identity) of amino acid residues in the training sequence being modified with changes sampled from a noninformative prior data distribution, such as a uniform distribution each possible type of amino acid residue is equally likely, the protein design engine 110 may add noise to the training sequence by changing the type (or identity) of the amino acid residues in the training sequence to alternatives that with a greater probability of occupying the corresponding position in the training sequence. Doing so may generate, for the noisy training sample, a noisy training sequence that is different from that of the original training sample but is nevertheless consistent with realistic protein sequences.

Where the informative prior distribution includes conditional atom dependencies in which atoms are confined to positions that preserve the chain-like conformation of protein molecules, the protein design engine 110 may generate the noisy training sample by modifying the training three-dimensional structure of the training protein molecule in accordance with the aforementioned conditional atom dependencies. For instance, in some cases, the modifications to the training three-dimensional structure of the training protein molecule may avoid changes in atomic positions that disrupt the chain-like conformation of the training protein molecule. In other words, instead of modifications that place atoms at arbitrary positions in three-dimensional space, which can result excessive distances between atoms from adjacent amino acid residues, the protein design engine 110 may limit the extent to which the positions of some atoms can change relative to that of other atoms in adjacent amino acid residues. As such, the incorporation of the informative prior data distribution may encourage the preservation of the chain-like conformation of the training protein molecule when generating the noisy training sample.

At 404, the protein design engine 110 may train, based at least on the training set, the protein design computation model 115 by at least adjusting one or more parameters of the protein design computation model 115 such that the protein design computation model 115 denoises the noisy training sample to recover the training sequence and the training three-dimensional structure of the training protein molecule. In some example embodiments, the protein design engine 110 may train, based on the training samples in the training set, the protein design computation model 115 to perform the generative task of co-designing the sequence and the three-dimensional structure of protein molecules. For example, in some cases, the protein design computation model 115 may be trained to generate the sequence and the three-dimensional structure of protein molecules by being trained to perform a reverse diffusion process in which the sequence and the three-dimensional structure of each noisy training sample are jointly denoised to recover the corresponding training protein molecule. In some cases, the reverse diffusion process may include recovering a training protein molecule through the gradual removal of noise from the sequence and the three-dimensional structure of the corresponding noisy training sample. For instance, in some cases, the protein design computation model 115 may be trained to remove a first portion of noise from the noisy training sample at a first step followed by a second portion of noise at a second step. In some cases, the training of the protein design computation model 115 may include adjusting one or more parameters (e.g., weights, biases, and/or the like) of the protein design computation model 115 to increase the fidelity of the joint denoising performed by the protein design computation model 115. That is, in some cases, the training of the protein design computation model 115 may include adjusting one or more parameters (e.g., weights, biases, and/or the like) of the protein design computation model 115 to decrease (or minimize) the difference between a training protein molecule and the one that is recovered by the protein design computation model 115 jointly denoising the sequence and the three-dimensional structure of the noisy training sample generated by corrupting the training protein molecule.

To further illustrate, let X₀ denote a training protein molecule and X denote the corrupted training sample generated therefrom. The forward diffusion process

$$q ( {X}_{1} , ... , {X}_{T} \vert {X}_{0} ) = q ( {X}_{1} \vert {X}_{0} ) {\prod }_{t = 2}^{T} q ( {X}_{t} \vert {X}_{t - 1} )$$

may be Markovian, which allows the recovery of the reverse process

$$q ( {X}_{t - 1} \vert {X}_{t} , {X}_{0} ) = \frac{q ( {X}_{t} \vert {X}_{t - 1} ) q ( {X}_{t - 1} \vert {X}_{0} )}{q ( {X}_{t} \vert {X}_{0} )}$$

The protein design computation model 115 is therefore trained to approximate the true denoising process \hat{X}₀=ϕ(Xₜ, t), which may be accomplished by at least minimizing the variational upper bound on the negative log likelihood as shown below.

$${L}_{elbo} ( {X}_{0} ) := {𝔼}_{q ( {X}_{0} )} [ {\underbrace{{D}_{KL} ( q ( {X}_{T} \vert {X}_{0} ) \text{\vert \vert} ( p ( {X}_{t} )}}_{{L}_{T} ( {X}_{0} )} + {\sum }_{t = 2}^{T} {E}_{q ( {X}_{0} )} {\underbrace{{D}_{KL} ( q ( {X}_{t - 1} \vert {X}_{t} , {X}_{0} ) \text{\vert \vert} ( {p}_{θ} ( {X}_{t - 1} \vert {X}_{t} )}}_{{L}_{t} ( {X}_{0} )} - {E}_{q ( {X}_{0} )} {\underbrace{log ( {p}_{θ} ( {X}_{0} \vert {X}_{1} )}}_{{L}_{1} ( {X}_{0} )} ]$$

In practice, the protein design computation model 115 may be trained to reduce (or minimize) each of the Lₜ (X₀) loss terms and the L₀ (X₀) loss term individually, for example, by sampling an arbitrary step t∈{1, T}. It should be appreciated that the loss term Lₜ (X₀) may approach 0 for any data distribution if the joint denoising is performed over sufficiently many steps. The latent variable distribution q may be defined to enable an efficient direct sampling of any step in the trajectory of the forward diffusion process.

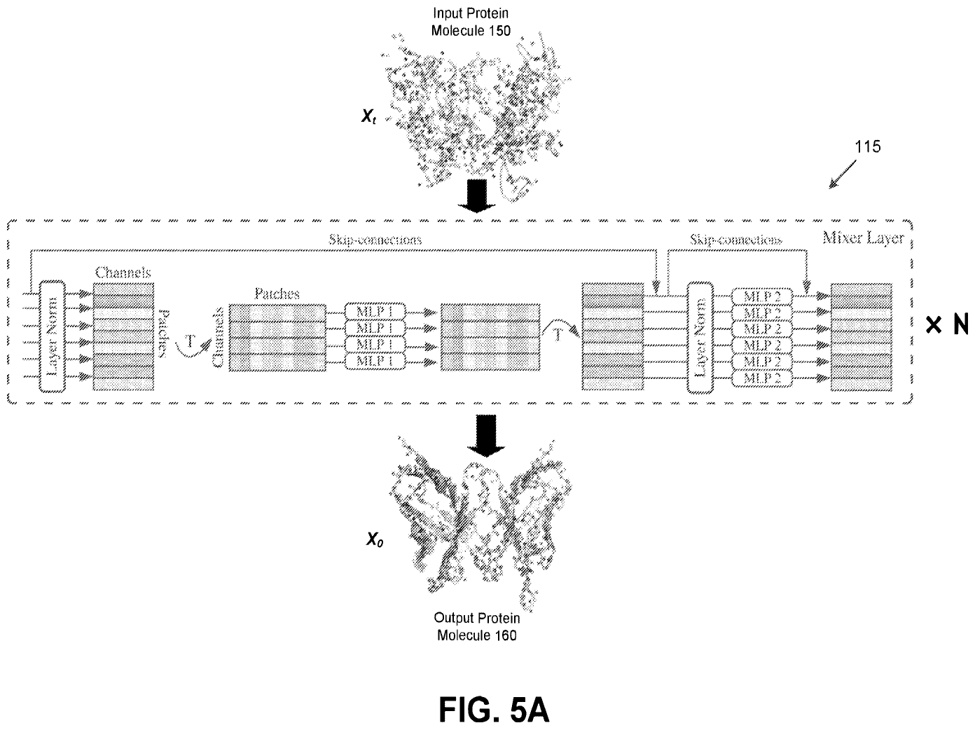

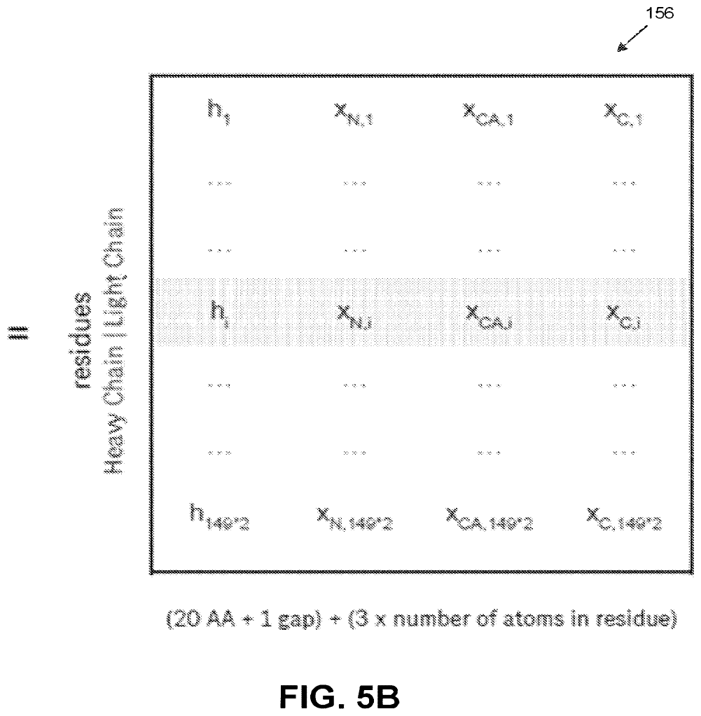

FIG. 5A depicts a schematic diagram illustrating an example of the architecture of the protein design computation model 115, in accordance with some example embodiments. As noted, in some example embodiments, the protein design computation model 115 may be implemented using one or more artificial neural networks (ANNs) such as multilayer perceptrons (MLPs) and/or the like. In some cases, the protein design computation model 115 may operate on the representation 156 of the input protein molecule 150 which, in some cases, may be a fixed-sized representation (e.g., fixed size tabular or matrix representation) of the input sequence 152 and the input three-dimensional structure 154. As described in more details below, doing so may reduce the architectural complexity of the protein design computation model. Moreover, the protein design computation model 115 may achieve equivariance (in three-dimensional structure) and invariance (in sequence) to group transformations (e.g., the rotations included in the special Euclidean group SE(3)) through frame averaging or another canonicalization technique.

Referring again to FIG. 5A, the protein design computation model 115 may include multiple blocks (of mixer layers), each of which containing multiple artificial neural networks (ANNs). In the example shown in FIG. 5A, the protein design computation model 115 may include an N quantity of blocks, each of which including two multilayer perceptrons (e.g., a first multilayer perceptron denoted MLP1 and a second multilayer perceptron denoted MLP2). In some cases, the blocks in the protein design computation model 115 may be applied consecutively on the columns and rows of the representation 156 of the input protein molecule 150. For example, in some cases, the representation 156 of the input protein molecule 150 is an r×c matrix X∈R^{r×c}. In some cases, the matrix X, which may be fixed in size, combines X^{pos} and X^{res} with one spot of the sequence occupying each row (e.g., 2×149 rows for a fixed-sized structural role based representation of the variable domains of immunoglobulin protein molecules). Accordingly, each row of the matrix X may include an encoding of the residue type and the positions (e.g., Cartesian coordinates (x, y, z)) of the atoms (e.g., the carbon (C), alpha carbon (C_{α}), nitrogen (N), beta carbon (C_{β}) backbone atoms) of the amino acid residue occupying the row. Given this formulation, the matrix X may have r=2×149 rows and c=21+4×3 columns. An example of the matrix X is shown in FIG. 5B, As shown in FIG. 5A, the blocks of the protein design computation model 115 may be applied consecutively to the columns c and the rows r in the manner below, with the normalization layer (LayerNorm in FIG. 5B) operating on each of the columns c and the rows r in accordance with the equations below

$$X {.}_{, j} = X {.}_{, j} + {W}_{2} ρ ( {W}_{1} LayerNorm ( X {.}_{, j} ) ) for all j ∈ [ c ]$$

$${X}_{i ,} . = {X}_{i ,} . + {W}_{4} ρ ( {W}_{3} LayerNorm ( {X}_{i ,} . ) ) for all i ∈ [ r ]$$

$$X {.}_{, j} = X {.}_{, j} + {LinearLayer}_{2} ( ρ ( {LinearLayer}_{1} ( LayerNorm ( X {.}_{, j} ) ) ) )$$

$${X}_{i ,} . = {X}_{i ,} . + {LinearLayer}_{4} ( ρ ( {LinearLayer}_{3} ( LayerNorm ( {X}_{i ,} . ) ) ) ) for all i ∈ [ r ] ,$$

wherein ρ denotes an activation function and the notation [k]=(1, . . . , k). The rows of the matrix X may be embedded in higher-dimensional vectors by the multilayer perceptrons (MLPs).

In some example embodiments, the protein design computation model 115 may achieve equivariance to Euclidean transformations (e.g., rotations and/or the like) of atoms through frame averaging. The frame averaging may be applied on the protein design computation model 115 in its entirety or on one or more of the N blocks individually. When frame averaging is applied to individual blocks of the protein design computation model 115, the high-dimensional embeddings may be split into three-dimensional sub-vectors for computing the frames. Moreover, because residue types are invariant to Euclidean transformations while atom position are equivariant, each of the N block's input and out vectors may be split in half, with one half encoding atomic positions being treated equivariantly and the other half encoding the type (or identity) of amino acid residues being treated invariantly.

When implemented in the manner described herein, the protein design computation model 115 may implicitly model pairwise interactions (between amino acid residues) by operating on the rows r and the columns c of the matrix X interchangeably. Accordingly, in some cases, the memory complexity of the protein design computation model 115 grows linearly with the quantity of amino acid residues instead of the usual quadratic complexity of conventional structure-based models. This reduction in memory complexity affords greater generative performance with a fixed runtime, parameter, and/or memory budget. In particular, the protein design computation model 115 having the architecture shown in FIG. 5A is able to use an order of magnitude more parameters with a smaller memory footprint during training while providing more efficient generation of protein molecules compared to conventional architectures like transformers and graph neural networks (GNNs). These advantages are shown in Table 1, which shows a comparison in the number of parameters, memory consumption during training (with a batch size of 4), memory consumption during generation protein molecules (of a batch of 10 examples for paired Observed Antibody Space (OAS)), and time taken to generate molecules for the protein design computation model 115 (AbDiffuser), a transformer, an equivariant graph neural network (EGNN), and a filter and augment graph neural network (FA-GNN).

| | | Memory | Memory | Generation |
|---|---|---|---|---|
| Model | Parameters | (training) | (generation) | time |
| Transformer | 84M | 14 GB | 15 GB | 3.2 min |
| EGNN | 39.3M | 78 GB | 16 GB | 22.6 min |
| FA-GNN | 9.4M | 75 GB | 38 GB | 9.5 min |
| AbDiffuser | 169M | 12 GB | 3 GB | 2.3 min |

In some example embodiments, invariance of the output sequence 152 to Euclidean transformations, such as the rotations and translations in the special Euclidean group SE(3) may be achieved by applying

$${X}^{res} = \frac{1}{\vert ℱ ( {X}^{pos} ) \vert} {\sum }_{( R , t ) ∈ ℱ ( {X}^{pos} )} ϕ ( {X}^{pos} R - 1 t , {X}^{res} )$$

when denoising residue types X^{res}. In addition to the output sequence 152 being invariant to Euclidean transformations (e.g., translations, rotations, and/or the like), the protein design computation model 115 may achieve equivariance to Euclidean transformations of the output three-dimensional structure 154 through averaging the output of the protein design computation model 115 not over the entire group of transformations but over a selected subset of group elements called frames. That is, the output three-dimensional structure 164 of the output protein molecule 160 may exhibit the same Euclidean transformations (e.g., translations, rotations, and/or the like) present in the input three-dimensional structure 154 of the input protein molecule 150. Frame averaging in this context may include the output three-dimensional structure 164 of the output protein molecule 160 being an average of the output the N blocks of the protein design computation model 115 operating on four frames, each of which having the input three-dimensional structure 154 of the input protein molecule 150 being oriented in one direction along two of the three principal axes of rotation. For example, in some cases, the N blocks of the protein design computation model 115 may operate a first frame in which the input three-dimensional structure 154 is oriented in one direction along a first principal axis of rotation, a second frame in which the input three-dimensional structure 154 is oriented in the opposite direction along the first principal axis of rotation, a third frame in which the input three-dimensional structure 154 is oriented in one direction along a second principal axis of rotation, and a fourth frame in which the input three-dimensional structure 154 is oriented in the opposite direction along the second principal axis of rotation. It should be appreciated that significant computational efficiency may be realized by averaging over the subset of group elements, such as the four aforementioned frames, instead of averaging over the entire group of transformations.

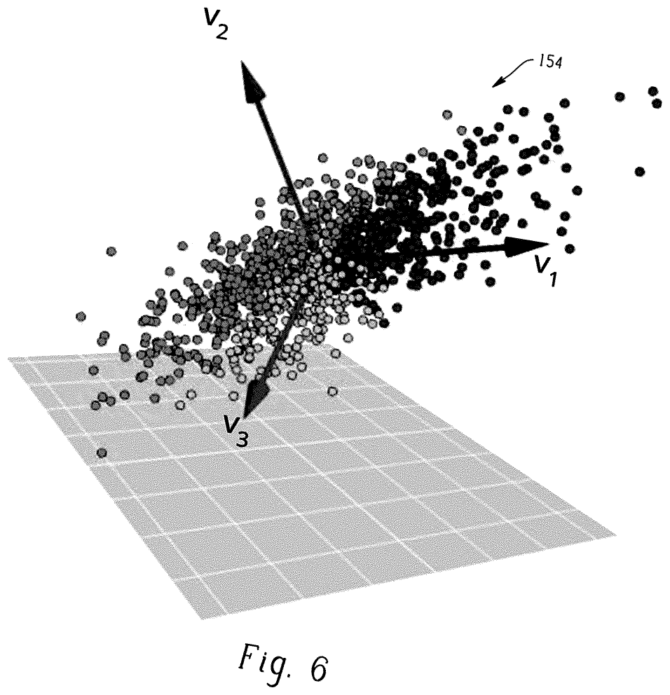

To further illustrate, FIG. 6 depicts a schematic diagram illustrating an example of the input three-dimensional structure 154 being oriented in three-dimensional space as a point cloud of atoms. In some cases, equivariance to Euclidean transformations, such as the rotations included in the special Euclidean group SE(3), may be achieved by averaging the outputs of the protein design computation model 115 not over the entire group of transformations but over a selected subset of group elements called frames F(X). For example, in some cases, equivariance to rotations (e.g., the SE (3) group) may be achieved as follows:

$${X}^{pos} = \frac{1}{\vert ℱ ( {X}^{pos} ) \vert} {\sum }_{( R , t ) ∈ ℱ ( {X}^{pos} )} ϕ ( {X}^{pos} R - 1 t , {X}^{res} ) {R}^{T} + 1 t wherein t = \frac{1}{n} {1}^{T} {X}^{pos}$$

is the centroid of the atoms in the input three-dimensional structure 154 and the four canonical rotation matrices R forming the four frames [symbol] (X^{pos})⊂SE(3) needed to achieve equivariance can be determined based on Principal Component Analysis.

In the example shown in FIG. 6, the four frames for achieving equivariance may be defined based on three unit-length eigenvectors v₁, v₂, v₃ corresponding to the eigenvalues λ₁, λ₂, λ₃ obtained from the eigen-decomposition of the covariance matrix C=(X^{pos}−1t)^{T} (X^{pos}−1t)∈R^{3×3}. Accordingly, the four frames may be defined as F(X^{pos})={([αv₁, βv₂, αv₁×βv₂], t)|α, β∈{−1,1}}. To respect the fixed chirality of certain protein molecules (such as those observed in humans) having non-identical mirror images, the operations of the protein design computation model 115 may be rendered equivariant with respect to special Euclidean group SE(3) and but not Euclidean group E(3), which also includes reflections. Accordingly, the four frames that are being averaged across excludes ones in which the input three-dimensional structure 154 is oriented in either direction along the third principal axis of rotation. Instead, the direction in which the input three-dimensional structure 154 is oriented along the third principal axis of rotation may be determined based on the right-hand rule as the cross product of the other two principal axes of rotation.

The example of the protein design computation model 115 shown in FIG. 5A, which includes the N-quantity of blocks (or mixer layers), is able to approximate any SE(3)-equivariant function when combined with frame averaging. Consider the example in which X is an n x m input matrix with bounded entries and the protein design computation model 115 is implemented with a multilayer perceptron (MLP) mixer backbone architecture ϕ(X)=c_{L}⋄r_{L-1}⋄ . . . ⋄c₃⋄r₂⋄c₁ (X), where the subscript denotes the layer, cₗ is a multilayer perceptron (MLP) operating independently on each column of the input matrix X, and rₗ is a multilayer perceptron (MLP) operating independently on each row of the input matrix X. If [symbol] is a bounded G-equivariant frame and if ϕ is a backbone model that is universal over any compact set, then combining ϕ with frame-averaging over a frame-finite domain (e.g., a domain in which the frame cardinality is finite) yields a G-equivariant universal model [symbol] . Since the special Euclidean group SE(3) frame is bounded and thus the domain is frame-finite, [symbol] may be shown as a universal approximator of any continuous SE(3) function by showing that the multilayer perceptron (MLP) mixer is a universal approximator over continuous functions of any compact set (or simply universal). That is, there exists a parametrization so that ϕ(X)=c_{L} ([z; vec(X)]) for some vector z. Given the universal approximation property of multilayer perceptrons (MLPs), it follows that the multilayer perceptron (MLP) mixer backbone model ϕ is also universal. Specifically, with more powerful multilayer perceptrons, such as the column multilayer perceptron cₗ and the row multilayer perceptron cᵣ, there exists a multilayer perceptron (MLP) that can approximate various operations on the input matrix X. Rather than increasing the dimensionality to n+1, the multilayer perceptron can assign some vector outside of the bounded domain where the input data lives, thus preserving a dimension of n such that a constant number of multilayer perceptron (MLP) mixer blocks are necessary to achieve the desired output.

To further illustrate, let V be a (n+1) x (n+1) unitary matrix whose first column is the constant vector 1 and set U=V_{:,1:} to be the n+1×n submatrix. The first multilayer perceptron in a block of the protein design computation model 115 (denoted MLP1 in FIG. 5A) may embed each column vector x isometrically to n+1 as follows: c₁ (x)=Uₓ. By construction, every column of the output matrix X′ is now orthogonal to 1. The network appends U to the left of the output matrix

$${X}^{′} and {\overbrace{( U X ; ... ; U X )}}^{xn - 1}$$

to the right. Appending to the right, which involves replicating each row multiple times, is accomplished by a linear row operation. Appending to the left is more involved and can be achieved by iteratively building U column by column, with each column being added using a row multilayer perceptron (MLP) to add one dimension with a constant value and a column multilayer perceptron (MLP) to project the new constant vector to the appropriate column U. Because the columns are guaranteed to be orthogonal to the constant vector, the column multilayer perceptron (MLP) is able to distinguish the newly introduced column from the ones already in play. Let X″ denote the result of the foregoing process.

In some cases, a column multilayer perceptron (MLP) projects each column of the (n+1)×(n+nm) matrix to n dimensions by multiplication with U^{T}. The output of this layer is X′″=[I; X; . . . ; X]. A row multilayer perceptron (MLP) then zeroes out all elements except those indexed by the one-hot encoding of residue types in the first n entries. This operation can be performed by a rectifier linear unit (ReLU) multilayer perceptron (MLP) with a single hidden layer. For example, if all entries in the input are smaller than b and the input is (onehot (i), x), the first linear layer adds b to only the entries [n+in: n+in +n) and shifts every element by −b using its bias. The latter permits the correct element to pass through the rectifier linear unit (ReLU) with an unchanged value while setting all other elements to zero. The output of this layer may be

$${X}_{i , :}^{′′′′} = ( {I}_{i , :} ; {\overbrace{( 0 ; ... ; 0 )}}^{xn - 1} ; {X}_{i , :} ; {\overbrace{( 0 ; ... ; 0 )}}^{xn - 1} ) .$$

Thereafter, a column multilayer perceptron (MLP) sums up all entries, with the output being a row vector (1; X_{1,:}^{T}; . . . ; Xₙ^{T})^{T} of length n+nm.

In some example embodiments, the protein design computation model 115 may include one or more projection layers that modifies the input three-dimensional structure 154 prior and/or subsequent to the updating of the input three-dimensional structure 154, for example, by the N-quantity of blocks (or mixer layers) of the protein design computation model 115. Atoms in a protein molecule abide by strong bond constraints including, for example, bond length, bond angle, and/or the like. While a machine learning model, such as a neural network, is capable of learning these constraints with a sufficient quantity of training data, doing so may require significant computational resources. As such, in some cases, the one or more projection layers of the protein design computation model 115 may impose one or more bond constraints by at least modifying the input three-dimensional structure 154 accordingly. For example, as shown in FIG. 2B, in some cases, the one or more projection layers may modify the input three-dimensional structure 154 to conform to the one or more bond constraints prior to the input three-dimensional structure 154 and the input sequence 152 undergo a joint denoising by the N-quantity of blocks (or mixer layers) of the protein design computation model 115. Furthermore, after the joint denoising of the input sequence 152 and the input three-dimensional structure 154 is complete, the one or more projection layers may again modify the input three-dimensional structure 154 to conform to the one or more bond constraints in order to generate the output three-dimensional structure 164 of the output protein molecule 160. As described in more details below, the modifications may include updating the positions of the backbone atoms and/or the positions of the side chain atoms forming each amino acid residue in the input three-dimensional structure 154 in order to conform to the one or more bond constraints.

In some example embodiments, the one or more projection layers may modify the positions of the backbone atoms in the input three-dimensional structure 154 based on a reference residue backbone in which the backbone atoms (e.g., carbon, α-carbon, nitrogen, and β-carbon) are positioned with idealized bond lengths and idealized bond angles therebetween. As noted, in some cases, this modification may be made prior and/or subsequent to the updating of the input three-dimensional structure 154. For example, in some cases, the one or more projection layers may determine one or more one or more transformations (e.g., roto-translation) that can be applied to the positions (e.g., Cartesian coordinates (x, y, z)) of the backbone atoms in the input three-dimensional structure 154 to minimize the distance (e.g., root mean squared deviation (RMSD)) between the atoms in the input three-dimensional structure 154 and the atoms in the reference residue backbone. Moreover, the one or more projection layers may modify the input three-dimensional structure 154 by at least applying the one or more transformations to positions (e.g., Cartesian coordinates (x, y, z)) of the backbone atoms in the input three-dimensional structure 154. In some cases, the one or more projection layers may also ensure that the distance between the carbon (C) and oxygen (O) backbone atoms in each amino acid residue satisfies one or more thresholds (e.g., 1.231 Angstroms) while remaining as close as possible (e.g., within a threshold distance) to the original position of the oxygen (O) atom. It should be appreciated that modifying the input three-dimensional structure 154 based on the reference residue backbone may introduce negligible difference (e.g., a root mean squared deviation (RMSD) of ˜5×10⁻³ Angstroms).

In some example embodiments, the one or more projection layers may modify the positions of the side chain atoms in the input three-dimensional structure 154 based on the side chain templates of the types of amino acid residue present in the input protein molecule 150. The actual identities of the amino acid residues forming the input protein molecule 150 remain unknown while the protein design computation model 115 is jointly denoising the input sequence 152 and the input three-dimensional structure 154. As such, the one or more projection layers may apply the side chain templates of the amino acid residues forming the input three-dimensional structure 154 subsequent to the joint denoising of the input sequence 152 and the input three-dimensional structure 154, at which point the identities of the amino acid residues forming the input protein molecules 150 are known. While the input sequence 152 and the input three-dimensional structure 154 is undergoing joint denoising, the N-quantity of blocks (or mixer layers) of the protein design computation model 115 may operate on a generic representation of the side chain of each amino acid residue. As described in more details below, the generic representation of the side chain of an amino acid residue may have sufficient degrees of freedom to account for the placement of the side chain atoms.

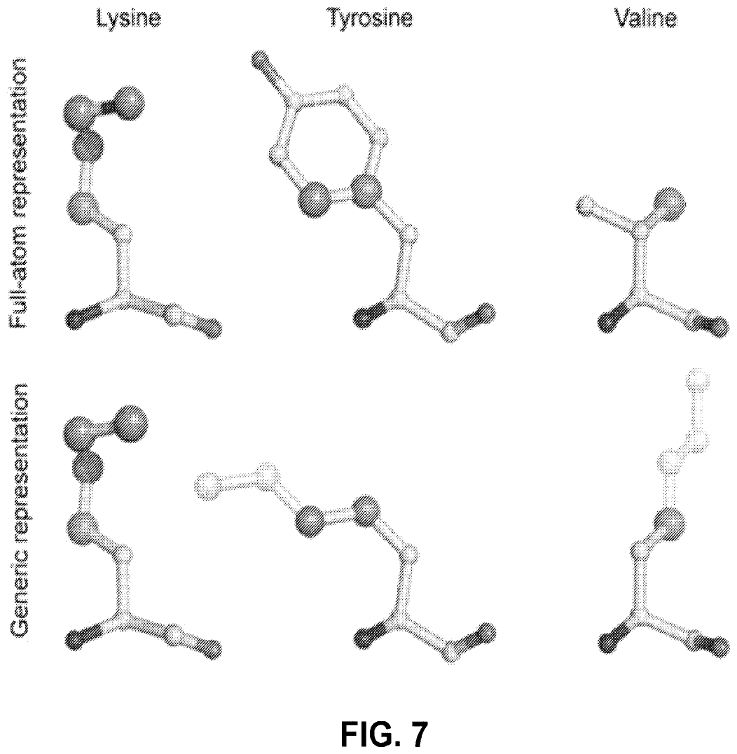

To further illustrate, FIG. 7 depicts schematic diagrams of the full-atom representation of the amino acid residue lysine, tyrosine, and valine, and examples of the corresponding generic representation, in accordance with some example embodiments. As shown in the full-atom representations in FIG. 7, the side chains of amino acid residues may contain up to four bonds capable of rotating about dihedral angles. These degrees of freedom may be captured by generic representations containing four pseudo-atoms that rotate about dihedral angles in the same way as the atoms in the full-atom representations. In cases where the full-atom representation of the side chain of an amino acid residue contains fewer than four atoms, FIG. 7 shows that the generic representation may reflect this reduction in degrees of freedom by setting the corresponding dihedral angles to 180°, which fixes these absent atoms to lie on a plane. Once the identities of the amino acid residues in the input protein molecule 150 are determined, the one or more projection layers may ensure that the bond length between the pseudo-atoms are consistent with bond constraints (e.g., 1.54 Angstroms). Furthermore, the one or more projection layers may extract the dihedral angles from the generic representations before applying these dihedral angles to the side chain templates of the specific types of amino acid residues identified as being a part of the input protein molecule 154.

In some example embodiments, the complexity of the joint denoising performed by the protein design computation model 115 may be reduced by at least incorporating an informative prior data distribution, such as positional amino acid residue frequencies, conditional atom dependencies, and/or the like. A noninformative prior data distribution, such as a uniform distribution or a Gaussian (normal) distribution, increases the complexity of the protein design computation model 115 at least because of the noninformative prior data distribution may bear little semblance to the posterior data distribution of protein molecules (or certain subsets thereof) the protein design computation model 115 is being trained to approximate. In cases where the protein design computation model 115 is trained to denoise protein molecules whose sequence and/or structure contain random noise sampled from a noninformative prior data distribution, the protein design computation model 115 may learn unrealistic modifications. Thus, in cases where the protein design computation model 115 is trained without the benefit of an informative prior data distribution, the protein design computation model 115 may learn a denoising diffusion function with unnecessary computational complexity. Contrastingly, when the training of the protein design computation model 115 incorporates an informative prior data distribution, the protein design computation model 115 may learn a less computationally complex denoising diffusion function such that the protein design computation model 115 is capable of operating faster and with greater computational efficiency.

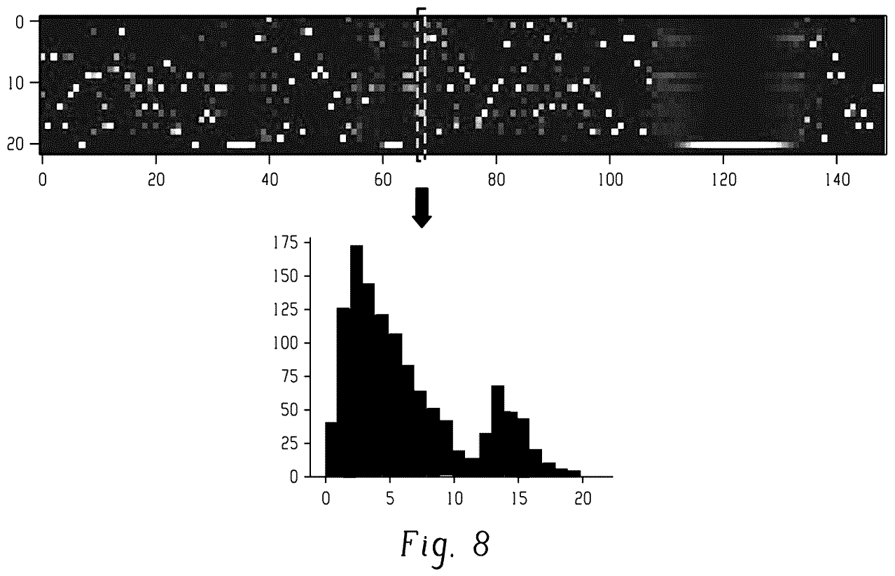

As noted, incorporating an informative prior data distribution may include generating training samples with the addition of noise sampled from the informative prior data distribution. In the case of an informative prior data distribution in the form of positional amino acid residue frequency, the training sequence of a noisy training sample may be generated with the addition of noise consistent with the positional amino acid residue frequency observed in protein molecules (or certain subsets thereof) such that the training sequence is formed by a permutation of amino acid residues and gap characters consistent with what is observed in protein molecules (or certain subsets thereof) rather than a random (or arbitrary) selection of amino acid residues. To further illustrate, FIG. 8 depicts a schematic diagram illustrating the positional amino acid residue frequency observed in a certain family of protein molecules, in this case the variable domain of immunoglobulin protein molecules (or antibodies). In the example shown in FIG. 8, each spot in the sequence of an immunoglobulin protein molecule (or antibody) may be assigned a particular structural role (e.g., 149 possible structural roles in the example shown). As shown in FIG. 8, an informative prior data distribution for positional amino acid residue frequency recognizes that, in nature, certain structural roles in an immunoglobulin protein molecule (or antibody) are more likely to be occupied by some types of amino acid residues than other types of amino acid residues. Contrastingly, a noninformative prior data distribution may assume a uniform (or equal) likelihood for every type of amino acid residue, in which case each structural role in the training sequence may be randomly assigned a type of amino acid residue or a gap character. Thus, in some cases, the positional amino acid residue frequency for immunoglobulin protein molecules (or antibodies) may be incorporated as an informative prior data distribution by at least modifying the type (or identity) of the amino acid residue occupying each positional role based on the corresponding frequencies rather than a randomly (or arbitrarily) selected amino acid residue.

In some example embodiments, instead of or in addition to positional amino acid residue frequency, the training of the protein design computation model 115 may also include an informative prior data distribution in the form of conditional atom dependencies. Conditional atom dependencies refer to the relative positions (or proximity) of atoms in adjacent amino acid residues. That is, protein molecules in nature assume a chain-like conformation, meaning that atoms in two (or more) adjacent amino acid residues are more likely to occupy proximate positions. Accordingly, an informative prior data distribution in the form of conditional atom dependencies respects the chain-like conformation of protein molecules by at least limiting the extent to which the positions of some atoms can change relative to that of other atoms in adjacent amino acid residues. For example, in some cases, the protein design engine 110 may generate, based at least on the three-dimensional structures of known protein molecules, an adjacency matrix specifying the conditional dependencies (and independencies) that exist between the positions of atoms in neighboring amino acid residues. Incorporating the informative data prior distribution in this case may include generating, based at least on the adjacency matrix, the training three-dimensional structure of one or more training protein molecules included in the training set for training the protein design computation model 115. For instance, in some cases, the training three-dimensional structure of a training protein molecule may be generated by modifying the positions (e.g., Cartesian coordinates (x, y, z)) of one or more atoms in accordance with the adjacency structure such that the training three-dimensional structure exhibits the chain-like conformation of protein molecules observed in nature.

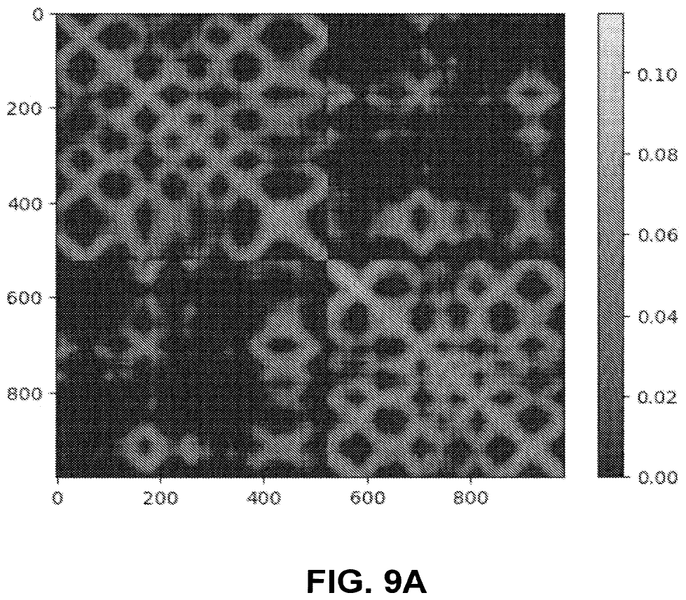

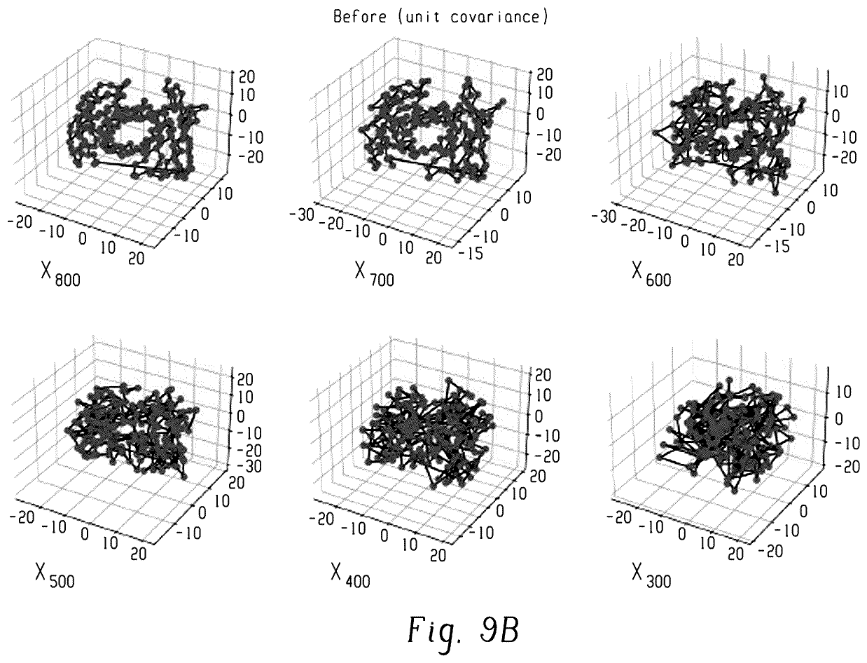

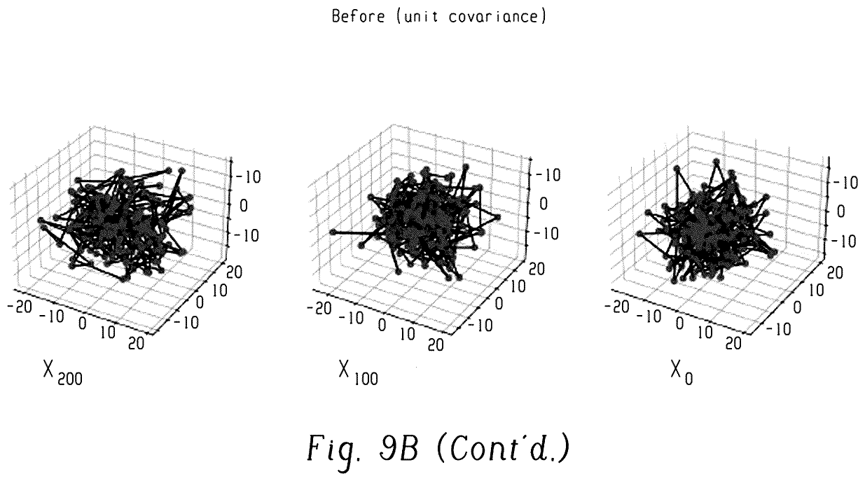

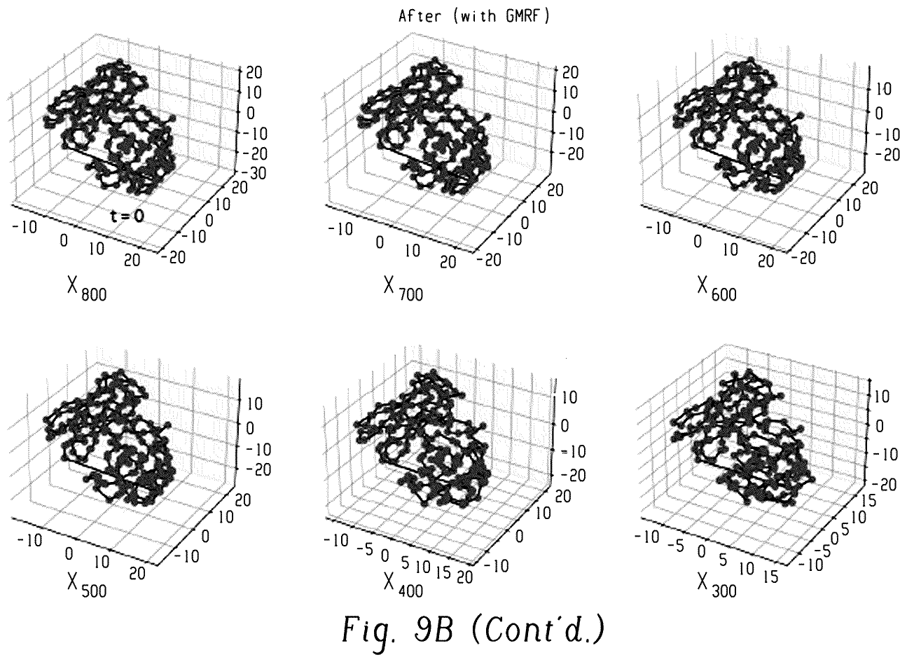

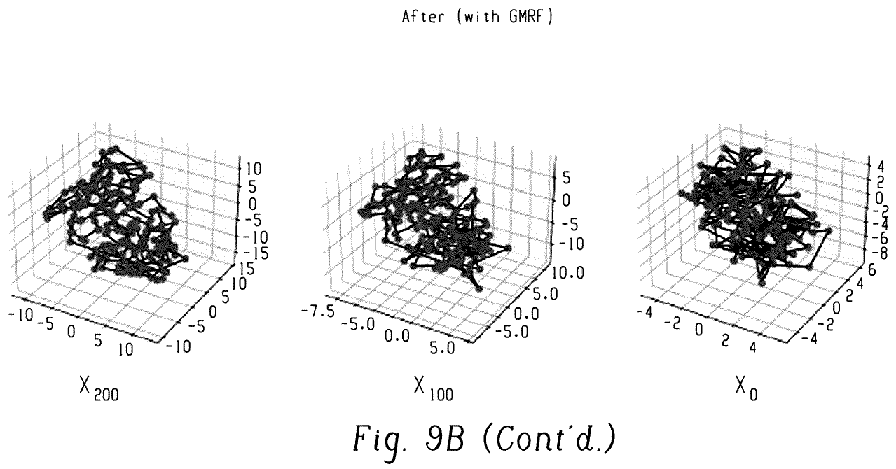

To further illustrate, FIG. 9A depicts an example of an adjacency matrix A for the atoms forming the backbone of immunoglobulin protein molecules (or antibodies), in accordance with some example embodiments. The example of the adjacency matrix A in FIG. 9A shows the dependencies between the positions of backbone atoms in the framework region of immunoglobulin protein molecules (or antibodies). Furthermore, the example of the adjacency matrix A in FIG. 9A shows the dependencies between the positions of backbone atoms in the heavy chain (top-left) and light chain (bottom-right) of immunoglobulin protein molecules (or antibodies).

In some cases, the conditional dependencies included in the adjacency matrix A may be estimated graphically, for example, by a Gaussian Markov Random Field (GMRF). In this context, the Gaussian Markov Random Field (GMRF) may correspond to a zero mean multivariate Gaussian [symbol] (0, Σ) over atomic positions (e.g., Cartesian coordinates (x, y, z)) whose inverse covariance matrix Σ⁻¹=L+aI is equal to a weighted sparse Laplacian matrix L=diag (A1)−A of the adjacency matrix A, with a being a small constant shifting the spectrum of the Laplacian matrix L to confer invertibility. The Gaussian Markov Random Field (GMRF) may operate under the assumption that positions (e.g., Cartesian coordinates (x, y, z)) of atoms in non-adjacent amino acid residues, which lack an interconnecting edge in the adjacency matrix A, are conditionally independent. Thus, the entry L_{i,j} in the Laplacian matrix L should be zero if the position of atom i is independent of the position of atom j where the positions of the neighboring atoms of atom i are fixed. In some cases, the Laplacian matrix L may be computed by minimizing the objective

$${min}_{L ∈ ℒ} tr ( {( {X}^{pos} )}^{T} L {X}^{pos} ) + f ( L )$$

in which X is an m×3 matrix containing the three-dimensional coordinates of an m quantity of atoms and ƒ (L) denotes some optional constraints enforceable upon the Laplacian matrix L (and in turn the adjacency matrix A). It should be appreciated the graphical representation of the conditional dependencies present in protein molecules conveys the sparsity of such relationships, which means that the Gaussian Markov Random Field (GMRF) estimation of the adjacency matrix A may be computed quickly and with little computational burden. FIG. 9B shows that the inclusion of conditional dependencies in atomic positions, for example, via Gaussian Markov Random Field (GMRF), reduces denoising complexity as the denoising process is able to recover chain-like protein conformations faster than in the absence of conditional dependencies (unit covariance).

In some example embodiments, the performance of the protein design computation model 115 may be evaluated by at least evaluating the output protein molecule 160 generated by the protein design computation model 115 jointly denoising the input sequence 152 and the input three-dimensional structure 154 of the input protein molecule 150 based on a variety of metrics. For example, in instances where the output protein molecule 160 is an immunoglobulin protein molecule (or an antibody), the output sequence 162 of the output protein molecule 160 may be evaluated for naturalness, similarity to the closest extant antibody, and stability. In this context, naturalness may be evaluated based on the inverse perplexity of the AntiBERTy model trained on a large corpus of antibody chain sequences. Similarity to the closest extant antibody may be determined by first identifying the closest training sequence in the training set with fast sequence alignment before the similarity between the two sequences are measured based on mean sequence identity (or fractional edit distance). Stability in this context may be determined by at least estimating the error in the folded three-dimensional structure with IgFold, with the resulting error used to rank the sequences on expected stability. The 90^{th} percentile of residue error may be taken as an estimate of the sequence fold stability, with the latter typically corresponding to the complementarity determining region (CDR) H3 and L3 loops. It should be appreciated that the complementarity determining region (CDR) H3 and L3 loops have the most influence over an antibody's functional properties.

To further verify that the output protein molecule 160, in this case an immunoglobulin protein molecule (or an antibody), additional structure-based metrics, such as complementarity determining region (CDR) hydrophobicity (CDR PSH), complementarity determining region (CDR) patches of positive charge (CDR PPC), complementarity determining region (CDR) patches of negative charge (CDR PNC), and symmetry of electrostatic charges of heavy and light chains (SFV CSP), may be used. It should be appreciated that the foregoing examples of structure-based metrics operate on the folded sequence (e.g., using IgFold) and take into account distance-aggregated structural properties of complementarity determining regions (CDRs) and their spatial vicinity, with significant deviations from reference values typically indicative of poor developability properties (e.g., expression, aggregation, non-specific binding, and/or the like). In some cases, the performance of the protein design computation model 115 may be evaluate based on the overall similarity of the generated and test-set distributions, which is measured as the deviation (e.g., Wasserstein distance) between the metrics of the sequences in the test set and the metrics of the generated sequences.

In some example embodiments, the output three-dimensional structure 164 of the output protein molecule 160 may be evaluated based on metrics known to correlate with free energy and root mean square deviation (RMSD). For example, in some cases, free energy ΔG (e.g., estimated using Rosetta) may be used to evaluate the stability of the output three-dimensional structure 164. While a lower free energy ΔG is typically associated with a higher stability, a disproportionately small energy achieved when a miss-formed three-dimensional structure collapses into a morph-less aggregate should still be avoided. Accordingly, the performance of the protein design computation model 115 in generating the output three-dimensional structure 164 may be evaluated based on the deviation (e.g., Wasserstein distance) between the free energies ΔG of the generated three-dimensional structures and those in the test set. In the case of root mean square deviation (RMSD), the output sequence 162 may be refolded (e.g., using IgFold) and the root mean square deviation (RMSD) between the refolded output sequence 162 and the output three-dimensional structure 164, in particular the root mean square deviation (RMSD) between the backbone atoms (e.g., the carbon (C), alpha carbon (C_{α}), nitrogen (N), beta carbon (C_{β}), and oxygen (O) backbone atoms), may be determined. The performance of the protein design computation model 115 in this case may be measured as a mean over the generated structures as it captures how well each generated structure matches that of the corresponding sequence.

Table 2 below enumerates detailed root mean square deviation (RMSD) for generated antibodies based on the Paired Observed Antibody Space (OAS) dataset. The root mean square deviation (RMSD) values in Table 2 compares the performance of conventional machine learning architectures equivariant graph neural network (EGNN) and filter and augment graph neural network (FA-GNN), and the protein design computation model 115 (AbDiffuser).

TABLE 2

| Model | Full ↓ | Fr ↓ | Fr. H ↓ | CDR H1↓ | CDR H2↓ | CDR H3↓ | Fr. L ↓ | CDR L1↓ | CDR L2↓ | CDR L3↓ |
|---|---|---|---|---|---|---|---|---|---|---|
| EGNN | 9.8231 | 9.3710 | 9.2929 | 13.1720 | 13.0032 | 10.3360 | 9.3918 | 14.6768 | 10.1584 | 10.4860 |
| EGNN (AHo) | 10.0628 | 9.4717 | 9.3552 | 13.1730 | 13.4611 | 12.2434 | 9.5314 | 15.3884 | 10.6975 | 11.0732 |
| EGNN (AHo & Cov.) | 9.4814 | 8.7581 | 8.6206 | 12.9454 | 13.2237 | 12.0939 | 8.8174 | 15.2841 | 10.0504 | 11.1167 |
| FA-GNN | 0.8617 | 0.5748 | 0.5093 | 0.6671 | 0.7438 | 2.2530 | 0.6157 | 0.8199 | 0.5946 | 1.1576 |
| FA-GNN (AHo) | 0.8321 | 0.4777 | 0.4618 | 0.6881 | 0.7867 | 2.2884 | 0.4860 | 0.9398 | 0.5053 | 1.1165 |
| FE-GNN (AHo & Cov.) | 0.8814 | 0.5934 | 0.5236 | 0.5968 | 0.6213 | 2.0788 | 0.5966 | 0.7907 | 0.4521 | 1.3536 |
| AbDiffuser (uniform prior) | 0.8398 | 0.5937 | 0.5742 | 0.7623 | 0.6705 | 1.8365 | 0.6095 | 0.8825 | 0.4795 | 1.0698 |
| AbDiffuser (no projection) | 11.1431 | 11.0062 | 10.8279 | 13.8692 | 14.4139 | 10.4367 | 11.1709 | 15.7536 | 11.5205 | 11.2404 |
| AbDiffuser (no Cov.) | 0.6302 | 0.4011 | 0.3826 | 0.4946 | 0.5556 | 1.6553 | 0.4169 | 0.5585 | 0.4321 | 0.8310 |
| AbDiffuser | 0.5230 | 0.3109 | 0.2862 | 0.3568 | 0.3917 | 1.5073 | 0.3322 | 0.4036 | 0.3257 | 0.7599 |
| AbDiffuser (side chains) | 0.4962 | 0.3371 | 0.3072 | 0.3415 | 0.3768 | 1.3370 | 0.3637 | 0.3689 | 0.3476 | 0.8173 |

Table 3 below enumerates detailed root mean square deviation (RMSD) for generated antibodies based on Trastuzumab mutant dataset. The root mean square deviation (RMSD) values in Table 2 compares the performance of conventional machine learning architectures equivariant graph neural network (EGNN), multi-channel equivariant attention network (MEAN), and filter and augment graph neural network (FA-GNN), and the protein design computation model 115 (AbDiffuser).

TABLE 3

| Model | Full ↓ | Fr ↓ | Fr. H ↓ | CDR H1 ↓ | CDR H2 ↓ | CDR H3 ↓ | Fr. L ↓ | CDR L1 ↓ | CDR L2 ↓ | CDR L3 ↓ |
|---|---|---|---|---|---|---|---|---|---|---|
| MEAN (Kong et al., 2023a) | 0.7792 | 0.3360 | 0.3045 | 0.4569 | 0.3359 | 2.9053 | 0.3645 | 0.4425 | 0.2490 | 0.6862 |
| EGNN (AHo & Cov.) | 9.2180 | 8.7818 | 8.5527 | 12.0018 | 12.5770 | 10.0308 | 8.9396 | 14.2269 | 9.5391 | 10.4077 |
| FA-GNN (AHo & Cov.) | 3.1800 | 3.3761 | 1.8529 | 0.6446 | 0.5223 | 2.0202 | 4.2721 | 0.5633 | 0.5376 | 3.4047 |
| AbDiffuser | 0.3822 | 0.2186 | 0.1669 | 0.3611 | 0.2737 | 1.1699 | 0.2610 | 0.1937 | 0.2006 | 0.6648 |
| AbDiffuser (side chains) | 0.4046 | 0.2686 | 0.2246 | 0.3861 | 0.3115 | 1.1191 | 0.3073 | 0.2242 | 0.2379 | 0.7122 |
| AbDiffuser (τ = 0.75) | 0.3707 | 0.2138 | 0.1615 | 0.3541 | 0.2709 | 1.1210 | 0.2563 | 0.1830 | 0.1946 | 0.6615 |
| AbDiffuser (s.c., τ = 0.75) | 0.3982 | 0.2729 | 0.2277 | 0.3914 | 0.2917 | 1.0624 | 0.3127 | 0.2492 | 0.2548 | 0.7131 |
| AbDiffuser (τ = 0.01) | 0.3345 | 0.2000 | 0.1463 | 0.3389 | 0.2723 | 0.9556 | 0.2430 | 0.1530 | 0.1792 | 0.6582 |
| AbDiffuser (s.c., τ = 0.01) | 0.6795 | 0.6168 | 0.5938 | 0.8161 | 0.7113 | 1.1550 | 0.6396 | 0.7938 | 0.7048 | 0.8395 |

Tables 2 and 3 provide per-region distances between the folded and optimized structure of generated sequences and generated structures. As shown, these distances correlate well with the overall root mean square distance. The results in Table 3 shows that the protein design computation model 115 (AbDiffuser) is able to model the complementarity determining region (CDR) H3 loop, which is the most important part of the antibody function-wise, with a high degree of precision. As shown in Tables 4 and 5 below, further optimization of the folded structures yielded modest changes in the three-dimensional structures generated by the protein design computation model 115 (AbDiffuser), evidencing the physical plausibility of the three-dimensional structures generated by the protein design computation model 115.

Table 4 below enumerates detailed root mean square (RMSD) for generated antibodies based on the Paired Observed Antibody Space (OAS) after optimization with Rosetta.

TABLE 4

| Model | Full ↓ | Fr ↓ | Fr. H ↓ | CDR H1 ↓ | CDR H2 ↓ | CDR H3 ↓ | Fr. L ↓ | CDR L1 ↓ | CDR L2 ↓ | CDR L3 ↓ |
|---|---|---|---|---|---|---|---|---|---|---|
| EGNN | 9.8129 | 9.3487 | 9.2647 | 13.2206 | 13.1699 | 10.4327 | 9.3722 | 14.8368 | 10.1526 | 10.6565 |
| EGNN (AHo) | 10.1364 | 9.5233 | 9.3961 | 13.3611 | 13.7014 | 12.4793 | 9.5918 | 15.6919 | 10.8115 | 11.3710 |
| EGNN (AHo & Cov.) | 9.5411 | 8.8202 | 8.6761 | 13.0186 | 13.3938 | 12.1843 | 8.8849 | 15.4368 | 10.1352 | 11.2356 |
| FA-GNN | 0.7817 | 0.4844 | 0.4228 | 0.5558 | 0.6293 | 2.1533 | 0.5205 | 0.7222 | 0.4617 | 1.0457 |
| FA-GNN (AHo) | 0.7767 | 0.4116 | 0.3918 | 0.6002 | 0.7031 | 2.2311 | 0.4228 | 0.8372 | 0.4054 | 1.0333 |
| FE-GNN (AHo & Cov.) | 0.7798 | 0.5061 | 0.4541 | 0.5485 | 0.5949 | 1.9846 | 0.5195 | 0.7121 | 0.3914 | 1.2032 |
| AbDiffuser (uniform prior) | 0.8122 | 0.5528 | 0.5300 | 0.7053 | 0.6184 | 1.8129 | 0.5704 | 0.8326 | 0.3914 | 1.0416 |
| AbDiffuser (no projection layer) | 10.9194 | 10.7255 | 10.5253 | 13.6499 | 15.0346 | 10.9846 | 10.9083 | 15.9310 | 11.6059 | 11.7446 |
| AbDiffuser (no Cov.) | 0.5867 | 0.3425 | 0.3206 | 0.4272 | 0.4848 | 1.6261 | 0.3606 | 0.4921 | 0.3296 | 0.7801 |
| AbDiffuser | 0.5068 | 0.2896 | 0.2642 | 0.3282 | 0.3708 | 1.4921 | 0.3110 | 0.3871 | 0.2611 | 0.7334 |
| AbDiffuser (side chains) | 0.4463 | 0.2751 | 0.2426 | 0.2764 | 0.3266 | 1.2869 | 0.3025 | 0.3187 | 0.2390 | 0.7533 |

Table 5 enumerates detailed root mean square distance (RMSD) for generated antibodies based on the Trastuzumab mutant dataset after optimization with Rosetta.

TABLE 5

| Model | Full ↓ | Fr ↓ | Fr. H ↓ | CDR H1 ↓ | CDR H2 ↓ | CDR H3 ↓ | Fr. L ↓ | CDR L1 ↓ | CDR L2 ↓ | CDR L3 ↓ |
|---|---|---|---|---|---|---|---|---|---|---|
| MEAN (Kong et al., 2023a) | 0.7412 | 0.3004 | 0.2718 | 0.5395 | 0.2909 | 2.7830 | 0.3261 | 0.3758 | 0.2593 | 0.6849 |
| EGNN (AHo & Cov.) | 9.2535 | 8.8170 | 8.5701 | 12.0330 | 12.7993 | 10.1256 | 8.9911 | 14.4588 | 9.7059 | 10.6565 |
| FA-GNN (AHo & Cov.) | 2.1631 | 2.2522 | 1.1541 | 0.6734 | 0.5783 | 2.0892 | 2.9101 | 1.4517 | 0.5797 | 2.1591 |
| AbDiffuser | 0.3692 | 0.2017 | 0.1415 | 0.3349 | 0.2474 | 1.1464 | 0.2479 | 0.1743 | 0.1589 | 0.6625 |
| AbDiffuser (side chains) | 0.4087 | 0.2755 | 0.2304 | 0.3632 | 0.3044 | 1.1065 | 0.3141 | 0.2957 | 0.1920 | 0.7217 |
| AbDiffuser (τ = 0.75) | 0.3584 | 0.1969 | 0.1358 | 0.3283 | 0.2459 | 1.1003 | 0.2434 | 0.1642 | 0.1513 | 0.6599 |
| AbDiffuser (s.c., τ = 0.75) | 0.3981 | 0.2747 | 0.2267 | 0.3615 | 0.2795 | 1.0497 | 0.3155 | 0.2939 | 0.2050 | 0.7151 |
| AbDiffuser (τ = 0.01) | 0.3210 | 0.1837 | 0.1202 | 0.3023 | 0.2464 | 0.9288 | 0.2306 | 0.1257 | 0.1285 | 0.6583 |
| AbDiffuser (s.c., τ = 0.01) | 0.6298 | 0.5661 | 0.5450 | 0.7135 | 0.6025 | 1.0870 | 0.5868 | 0.6878 | 0.6012 | 0.8720 |

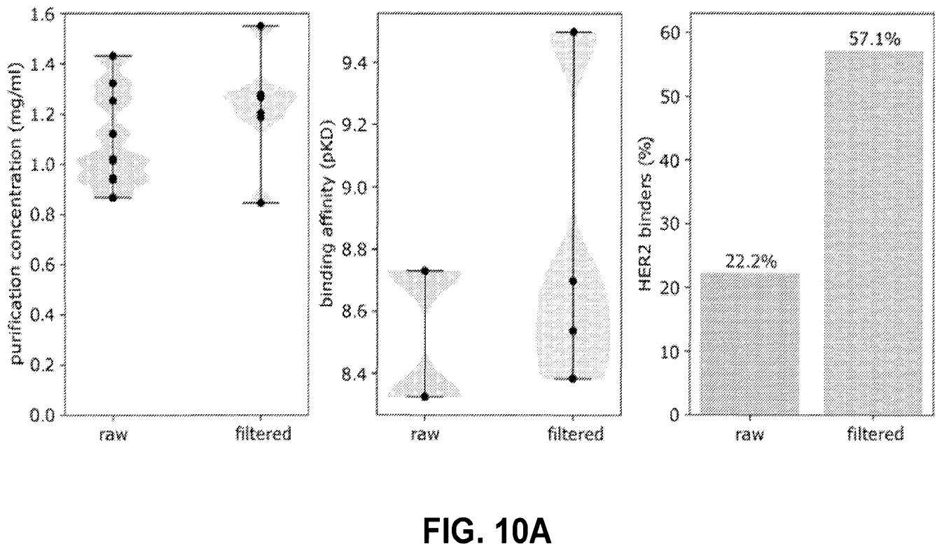

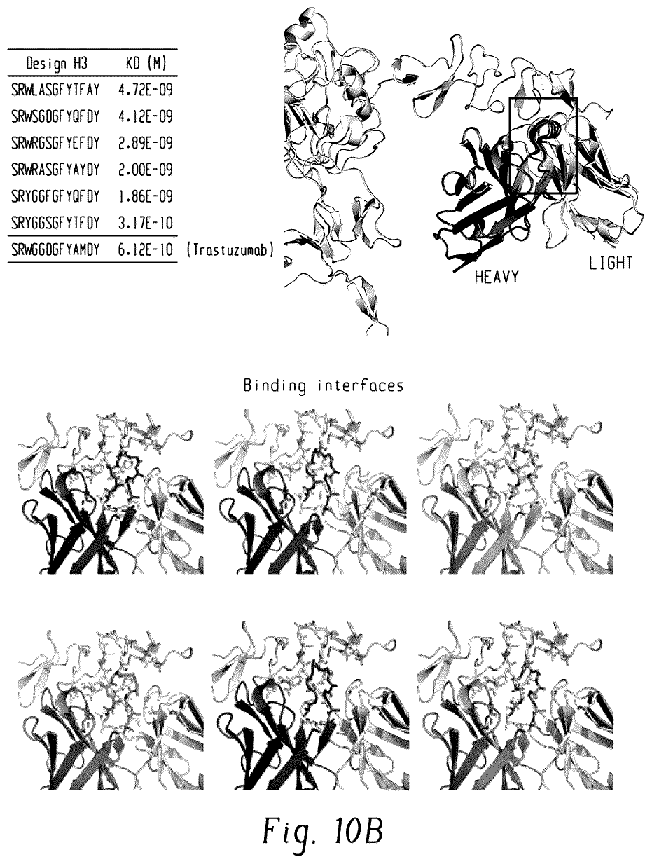

The performance of the protein design computation model 115 is also evaluated with in vitro experiments. For example, in some cases, the protein design computation model 115 is applied to mutate Trastuzumab such that the resulting antibody is still capable of binding to the target molecule human epidermal growth factor receptor 2 (HER2). FIG. 10A shows the results of in vitro validation of the Trastuzumab mutants generated by the protein design computation model 115 in terms of expression (left), binding affinity (center), and binding rate (right). The “raw” column corresponds to randomly selected antibodies generated by the protein design computation model 115 whereas the “filtered” column corresponds to designs that have undergone further in silico screening for certain desirable properties (e.g., by the analysis engine 120). FIG. 10B shows the structure of the target molecule human epidermal growth factor receptor 2 (HER2) as well as the three-dimensional structures of the binders generated by the protein design computation model 115. As shown in FIG. 10B, the protein design computation model 115 may learn to redesign certain portions of the binding interface while maintaining binding affinity.

As shown in FIG. 10A, every design generated by the protein design computation model 115 was expressed and purified successfully (average concentration of 1.25 milligrams per milliliter) and an average of 37.5% of the designs were confirmed as binders (with a dissociation constant range pKD ∈[8.32, 9.50] and an average of 8.70). Binding rate was improved (from 22.2% to 57.1%) when the “raw” designs generated by the protein design computation model 115 are filtered to exclude those in the bottom 25^{th} quantile in metrics such as naturalness, root mean square deviation (RMSD), and/or the like) and identified as non-binders (by a classifier trained to distinguish between binders and non-binders). The increase in binding indicates that metrics such as naturalness and root mean square deviation (RMSD) do correlate with desirable in vitro characteristics. The best binder came from the filtered set and exhibited a higher dissociation constant (pKD of 9.50) than Trastuzumab while differing in 4 positions in the complementarity determining region (CDR) H3 loop.

In contrast to existing methodologies for mutant design, the binders generated by the protein design computation model 115 achieved a higher binding affinity (pKD of 9.50 instead of 9.03). Furthermore, the protein design computation model 115 described herein is able to achieve the higher affinity binder more efficiently, with fewer wet lab resources. For example, existing methodologies use a simple sequence in-filing model trained on a large set of generic antibodies whereas various implementations of the protein design computation model 115 described herein may be trained on binders. Moreover, existing methodologies generated 440k candidate sequences that were then filtered in the wet-lab using high-throughput screening to identify 4k binders, out of which 421 binders were selected to be tested using precise surface plasmon resonance (SPR) assays. Contrastingly, sixteen samples generated by the protein design computation model 115 were tested in the web lab, meaning that the approach described herein required 26× fewer precise surface plasmon resonance (SPR) experiments to identify better binders.

Table 6 below enumerates the edit distances of the generated binders to the closest binders and non-binders. The task of generating binders is especially challenging because, as in the case of mutating Trastuzumab, some binders may be as few as a single edit (or difference in the sequence amino acid residues) away from non-binders. That is, the edit distance between a binding sequence and a nonbinding sequence is not necessarily greater than the edit distance between two different binding (or nonbinding) sequences. In other words, making fewer edits to a known binder, such as Trastuzumab, does not necessarily preserve the binding affinity of the known binder. Instead, the protein design computation model 115 is implicitly trained to recognize features associated with binding affinity and to preserve these features when generating the output sequence 162 through a joint denoising of the input sequence 152 and the input three-dimensional structure 164. This phenomenon is shown in Table 6, with most of the binders generated by the protein design computation model 115 having no more than two edits (or two different amino acid residues) relative to the closest non-binders. However, it should be appreciated that the protein design computation model 115 is able to learn critical features in the sequences of binders as well as non-binders, such that the protein design computation model 115 is able to mutate a binder, in this case Trastuzumab, with changes that do not disrupt binding affinity.

TABLE 6

| | Binder | Num. closest | Non-binder | Num. closest | |
|---|---|---|---|---|---|
| Generated binder | dist. | binders | dist. | non-binders | KD↓ |
| SRYGSSGFYQFTY | 2 | 2 | 2 | 2 | 5.35e−08 |
| SRWLASGFYTFAY | 1 | 1 | 2 | 2 | 4.72e−09 |
| SRWSGDGFYQFDY | 1 | 1 | 2 | 3 | 4.12e−09 |
| SRWRGSGFYEFDY | 1 | 1 | 2 | 3 | 2.89e−09 |
| SRWRASGFYAYDY | 1 | 2 | 3 | 19 | 2.00e−09 |
| SRYGGFGFYQFDY | 2 | 3 | 2 | 2 | 1.86e−09 |
| SRYGGSGFYTFDY | 2 | 8 | 2 | 2 | 3.17e−10 |

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:
Item 1: A computer-implemented method, comprising: receiving an input protein molecule including an input sequence and an input three-dimensional structure, the input sequence including a plurality of residues, and the input three-dimensional structure including a position of a plurality of atoms forming each residue included in the input sequence; generating a representation of the input protein molecule including the input sequence and the input three-dimensional structure; and applying a protein design computation model to generate, based at least on the representation of the input protein molecule, an output protein molecule, the protein design computation model generating the protein molecule by at least performing a joint denoising of the input sequence and the input three-dimensional structure to generate an output sequence and an output three-dimensional structure of the output protein molecule.
Item 2: The method of Item 1, wherein the representation of the input protein molecule is a matrix having a plurality of rows, and wherein each row in the matrix corresponds to a spot in the input sequence.
Item 3: The method of claim 2, wherein the matrix includes one column populated by an encoding of a type of residue occupying each spot in the input sequence, and wherein the matrix further includes a column populated by one or more coordinates for each possible atom forming a residue.
Item 4: The method of any of Items 1 to 3, wherein the representation of the input protein molecule is a fixed size representation having a same quantity of rows and/or a same quantity of columns for different length input sequences.
Item 5: The method of any of Items 1 to 4, further comprising: generating the representation of the input protein molecule by at least assigning, to each residue included in the input sequence, an integer position corresponding to a structural role of the residue.
Item 6: The method of Item 5, wherein the representation of the input protein molecule includes a gap character for each integer position where the input sequence fails to include a residue having a corresponding structural role.
Item 7: The method of claim 6, further comprising: generating the representation of the input protein molecule by at least determining, based at least on a first position of each atom in one or more adjacent residues, a second position of each nonexistent atom associated with the gap character.
Item 8: The method of any of Items 1 to 7, wherein the protein design computation model jointly denoises the input sequence and the input three-dimensional structure over a plurality of successive denoising steps.
Item 9: The method of any of Items 1 to 8, wherein the protein design computation model jointly denoises the input sequence and the input three-dimensional structure by at least removing a first portion of noise at a first step before removing a second portion of noise at a second step.
Item 10: The method of any of Items 1 to 9, wherein the protein design computation model generates the output sequence to be invariant to special Euclidean group SE(3) transformations and the output three-dimensional structure to be equivariant to special Euclidean group SE(3) transformations.
Item 11: The method of any of Items 1 to 10, wherein the protein design computation model jointly denoises a plurality of frames in which each frame corresponds to the input three-dimensional structure being oriented in one of two possible directions along each of two principal axes of rotation about a centroid of the input three-dimensional structure, and wherein the protein design computation model generates the output three-dimensional structure by at least averaging a result of jointly denoising the plurality of frames.
Item 12: The method of Item 11, wherein the plurality of frames includes a first frame in which the input three-dimensional structure is oriented in one direction along a first principal axis of rotation, a second frame in which the input three-dimensional structure is oriented in an opposite direction along the first principal axis of rotation, a third frame in which the input three-dimensional structure is oriented in one direction along a second principal axis of rotation, and a fourth frame in which the input three-dimensional structure is oriented in the opposite direction along the second principal axis of rotation.
Item 13: The method of Item 12, wherein the protein design computation model determines, based at least on a first direction of the input three-dimensional structure along the first principal axis of rotation and a second direction of the input three-dimensional structure along the second principal axis of rotation, a third direction of the input three-dimensional structure along a third principal axis of rotation.
Item 14: The method of any of Items 1 to 13, wherein the protein design computation model includes a plurality of blocks, wherein each block of the protein design computation model includes a first multilayer perceptron (MLP) and a second multilayer perceptron (MLP), and wherein the plurality of blocks are applied consecutively to the representation of the input protein molecule.
Item 15: The method of any of Items 1 to 14, wherein the protein design computation model further includes a projection layer, and wherein the projection layer modifies, based on one or more bond constraints, the input three-dimensional structure prior and/or subsequent to the joint denoising.
Item 16: The method of Item 15, wherein the one or more bond constraints are imposed based on a reference residue backbone comprising a plurality of rigid atoms with fixed bond lengths and fixed bond angles.
Item 17: The method of Item 16, wherein the projection layer modifies the input three-dimensional structure by at least determining one or more transformations that minimize a distance between the plurality of rigid atoms in the reference residue backbone and one or more backbone atoms in the input three-dimensional structure, and applying the one or more transformations to align the input three-dimensional structure to the reference residue backbone.
Item 18: The method of any of Items 15 to 17, wherein the protein design computation model jointly denoises generic side chains comprising pseudo atoms having a same degrees of freedom as atoms in side chains of actual residues, and wherein the one or more bond constraints are imposed by at least replacing, subsequent to the joint denoising, the generic side chains with a side chain template of each type of residue forming the input three-dimensional structure.
Item 19: The method of Item 18, wherein the projection layer modifies the input three-dimensional structure subsequent to the joint denoising by at least applying a dihedral angle between one or more pseudo atoms in the generic side chain template to a corresponding side chain template.
Item 20: The method of any of Items 1 to 19, further comprising: determining an informative prior data distribution corresponding to a generative task of the protein design computation model; and incorporating the informative prior data distribution by at least generating, based at least on the informative prior data distribution, one or more training samples for the protein design computation model.
Item 21: The method of Item 20, wherein the generative task of the protein design computation model includes generating one or more protein molecules from a specific protein family.
Item 22: The method of any of Items 20 to 21, wherein the informative prior data distribution includes a positional residue frequency specifying a likelihood of different types of residues occupying each spot in a protein sequence.
Item 23: The method of any of Items 20 to 22, wherein the informative prior data distribution includes a conditional atom dependencies specifying a relative position of atoms in adjacent residues.
Item 24: The method of any of Items 20 to 23, wherein the one or more training samples are generated to by at least adding, to one or more training protein molecules, noise sampled from the informative prior data distribution.
Item 25: The method of Item 24, wherein the noise includes one or more modifications to at least one of a residue type and atomic positions.
Item 26: The method of any of Items 24 to 25, wherein the one or more training samples are generated by a forward diffusion process that includes an incremental addition of the noise sampled from the informative prior data distribution.
Item 27: The method of any of Items 1 to 26, wherein the input protein molecule exhibits one or more desirable properties, and wherein the protein design computation model generates the output protein molecule to also exhibit the one or more desirable properties.
Item 28: The method of Item 27, wherein the one or more desirable properties include expression, binding affinity towards a target molecule, specificity, stability, non-immunogenicity, human-ness, and/or lack of self-association.
Item 29: A system, comprising: at least one data processor; and at least one memory storing instructions, which when executed by the at least one data processor, result in operations comprising the method of any of Items 1 to 28.
Item 30: A non-transitory computer readable medium storing instructions, which when executed by at least one data processor, result in operations comprising the method of any of Items 1 to 28.

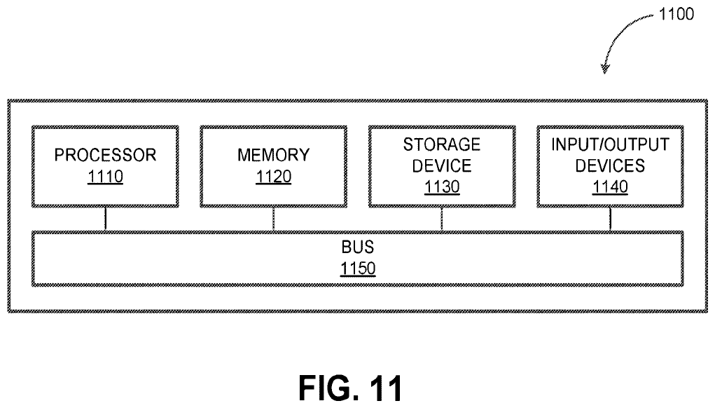

FIG. 11 depicts a block diagram illustrating an example of computing system 1100, in accordance with some example embodiments. Referring to FIGS. 1-11, the computing system 1100 may be used to implement the protein design engine 110, the analysis engine 120, the client device 130, and/or any components therein.

As shown in FIG. 11, the computing system 1100 can include a processor 1110, a memory 1120, a storage device 1130, and input/output devices 1140. The processor 1110, the memory 1120, the storage device 1130, and the input/output devices 1140 can be interconnected via a system bus 1150. The processor 1110 is capable of processing instructions for execution within the computing system 1100. Such executed instructions can implement one or more components of, for example, the protein design engine 110, the analysis engine 120, the client device 130, and/or the like. In some example embodiments, the processor 1110 can be a single-threaded processor. Alternatively, the processor 1110 can be a multi-threaded processor. The processor 1110 is capable of processing instructions stored in the memory 1120 and/or on the storage device 1130 to display graphical information for a user interface provided via the input/output device 1140.

The memory 1120 is a computer readable medium such as volatile or non-volatile that stores information within the computing system 1100. The memory 1120 can store data structures representing configuration object databases, for example. The storage device 1130 is capable of providing persistent storage for the computing system 1100. The storage device 1130 can be a floppy disk device, a hard disk device, an optical disk device, or a tape device, or other suitable persistent storage means. The input/output device 1140 provides input/output operations for the computing system 1100. In some example embodiments, the input/output device 1140 includes a keyboard and/or pointing device. In various implementations, the input/output device 1140 includes a display unit for displaying graphical user interfaces.

According to some example embodiments, the input/output device 1140 can provide input/output operations for a network device. For example, the input/output device 1140 can include Ethernet ports or other networking ports to communicate with one or more wired and/or wireless networks (e.g., a local area network (LAN), a wide area network (WAN), the Internet).

In some example embodiments, the computing system 1100 can be used to execute various interactive computer software applications that can be used for organization, analysis and/or storage of data in various formats. Alternatively, the computing system 1100 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1140. The user interface can be generated and presented to a user by the computing system 1100 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions, which when execute by the at least one data processor, result in operations comprising:
receiving an input protein molecule including an input sequence and an input three-dimensional structure,
the input sequence including a plurality of residues, and
the input three-dimensional structure including a position of a plurality of atoms forming each residue included in the input sequence;
generating a representation of the input protein molecule including the input sequence and the input three-dimensional structure; and
applying a protein design computation model to generate, based at least on the representation of the input protein molecule, an output protein molecule,
the protein design computation model generating the protein molecule by at least performing a joint denoising of the input sequence and the input three-dimensional structure.

2. The system of claim 1, wherein the representation of the input protein molecule is a matrix having a plurality of rows, and wherein each row in the matrix corresponds to a position in the input sequence.

3. The system of claim 2, wherein the matrix includes one column populated by an encoding of a type of residue occupying each position in the input sequence, and wherein the matrix further includes a column populated by one or more coordinates for each possible atom forming a residue.

4. The system of claim 1, wherein the representation of the input protein molecule is a fixed size representation having a same quantity of rows and/or a same quantity of columns for different length input sequences.

5. The system of claim 1, wherein the operations further comprise:
generating the representation of the input protein molecule by at least assigning, to each residue included in the input sequence, an integer position corresponding to a structural role of the residue.

6. The system of claim 5, wherein the representation of the input protein molecule includes a gap character for each integer position where the input sequence fails to include a residue having a corresponding structural role.

7. The system of claim 6, wherein the operations further comprise:
generating the representation of the input protein molecule by at least determining, based at least on a first position of each atom in one or more adjacent residues, a second position of each nonexistent atom associated with the gap character.

8. The system of claim 1, wherein the protein design computation model jointly denoises the input sequence and the input three-dimensional structure over a plurality of successive denoising steps.

9. The system of claim 1, wherein the protein design computation model jointly denoises the input sequence and the input three-dimensional structure by at least removing a first portion of noise at a first step before removing a second portion of noise at a second step.

10. The system of claim 1, wherein the protein design computation model generates the sequence of the output molecule to be invariant to special Euclidean group SE(3) transformations and the three-dimensional structure of the output molecule to be equivariant to special Euclidean group SE(3) transformations.

11. The system of claim 1, wherein the protein design computation model jointly denoises a plurality of frames in which each frame corresponds to the input three-dimensional structure being oriented in one direction along a principal axis of rotation about a centroid of the input three-dimensional structure, and wherein the protein design computation model generates the three-dimensional structure of the output molecule by at least averaging a result of jointly denoising the plurality of frames.

12. The system of claim 11, wherein the plurality of frames includes a first frame in which the input three-dimensional structure is oriented in one direction along a first principal axis of rotation, a second frame in which the input three-dimensional structure is oriented in an opposite direction along the first principal axis of rotation, a third frame in which the input three-dimensional structure is oriented in one direction along a second principal axis of rotation, and a fourth frame in which the input three-dimensional structure is oriented in the opposite direction along the second principal axis of rotation.

13. The system of claim 12, wherein the protein design computation model determines, based at least on a direction of the input three-dimensional structure along the first principal axis of rotation and a direction of the input three-dimensional structure along the second principal axis of rotation, a direction of the input three-dimensional structure along a third principal axis of rotation.

14. The system of claim 1 to 13, wherein the protein design computation model includes a plurality of blocks, wherein each block of the protein design computation model includes one or more multilayer perceptrons (MLP), and wherein the plurality of blocks are applied consecutively to the representation of the input protein molecule.

15. The system of claim 1, wherein the protein design computation model includes a projection layer that modifies, based on one or more bond constraints, the input three-dimensional structure.

16. The system of claim 15, wherein the one or more bond constraints are imposed based on a reference residue backbone comprising a plurality of rigid atoms with fixed bond lengths and fixed bond angles.

17. The system of claim 16, wherein the projection layer modifies the input three-dimensional structure by at least
determining one or more transformations that minimize a distance between the plurality of rigid atoms in the reference residue backbone and one or more backbone atoms in the input three-dimensional structure, and
applying the one or more transformations to align the input three-dimensional structure to the reference residue backbone.

18. The system of claim 15, wherein the protein design computation model jointly denoises, for each residue of the plurality of residues in the input sequence, a generic sidechains comprising pseudo atoms having a same degrees of freedom as atoms in a sidechains of an actual residues, and wherein the one or more bond constraints are imposed by at least replacing the generic sidechains with a sidechain template of a corresponding type of residue.

19. The system of claim 18, wherein the projection layer modifies the input three-dimensional structure subsequent to the joint denoising by the protein design computation model by at least applying a dihedral angle between one or more pseudo atoms in the generic sidechain to the sidechain template the corresponding type of residue.

20. The system of claim 1, wherein the operations further comprise:
determining a prior data distribution corresponding to a generative task of generating one or more protein molecules from a specific protein family; and
incorporating the prior data distribution by at least generating, based at least on the prior data distribution, one or more training samples for the protein design computation model.

21. (canceled)

22. The system of claim 20, wherein the prior data distribution includes a positional residue frequency specifying a likelihood of different types of residues occupying each position in a protein sequence.

23. The system of claim 20, wherein the prior data distribution includes a conditional atom dependency specifying a relative position of atoms in adjacent residues.

24. The system of claim 20, wherein the one or more training samples are generated to by at least adding, to one or more training protein molecules, noise sampled from the prior data distribution.

25. The system of claim 24, wherein the noise includes one or more modifications to at least one of a residue type and atomic positions.

26. The system of claim 24, wherein the one or more training samples are generated by a forward diffusion process that includes an incremental addition of the noise sampled from the prior data distribution.

27. The system of claim 1, wherein the input protein molecule exhibits a property, and wherein the protein design computation model generates the output protein molecule to exhibit a different property and/or to modify a magnitude of a same property present in the output protein molecule.

28. The system of claim 27, wherein the property includes one or more of expression, binding affinity towards a target molecule, specificity, stability, non-immunogenicity, human-ness, and lack of self-association.

29. A computer-implemented method, comprising:
receiving an input protein molecule including an input sequence and an input three-dimensional structure, the input sequence including a plurality of residues, and the input three-dimensional structure including a position of a plurality of atoms forming each residue included in the input sequence;
generating a representation of the input protein molecule including the input sequence and the input three-dimensional structure; and
applying a protein design computation model to generate, based at least on the representation of the input protein molecule, an output protein molecule, the protein design computation model generating the protein molecule by at least performing a joint denoising of the input sequence and the input three-dimensional structure.

30. A non-transitory computer readable medium storing instructions, which when executed by at least one data processor, result in operations comprising:
receiving an input protein molecule including an input sequence and an input three-dimensional structure, the input sequence including a plurality of residues, and the input three-dimensional structure including a position of a plurality of atoms forming each residue included in the input sequence;
generating a representation of the input protein molecule including the input sequence and the input three-dimensional structure; and
applying a protein design computation model to generate, based at least on the representation of the input protein molecule, an output protein molecule, the protein design computation model generating the protein molecule by at least performing a joint denoising of the input sequence and the input three-dimensional structure.

31. The system of claim 1, wherein generating the output protein molecule comprises generating a sequence of the output protein molecule or a three-dimensional structure of the output protein molecule
